(19) United States
(12) Patent Application Publication
(10) Pub. No.: US 2024/0010731 A1
(43) Pub. Date: Jan. 11, 2024

(54) COMBINATION PRODUCT FOR THE TREATMENT OF CANCER

(71) Applicants: Debiopharm International S.A., Lausanne (CH); Merck Patent GmbH, Darmstadt (DE); Pfizer Inc., New York, NY (US)

(72) Inventors: Grégoire Vuagniaux, Lausanne (CH); Norbert Wiedemann, Lausanne (CH); Claudio Zanna, Lausanne (CH)

(73) Assignees: Debiopharm International S.A., Lausanne (CH); Merck Patent GmbH, Darmstadt (DE); Pfizer Inc., New York, NY (US)

(21) Appl. No.: 18/328,707

(22) Filed: Jun. 2, 2023

Related U.S. Application Data
(63) Continuation of application No. 16/757,178, filed on Apr. 17, 2020, now abandoned.
(60) Provisional application No. 62/574,384, filed on Oct. 19, 2017.

(30) Foreign Application Priority Data
Aug. 31, 2018 (EP) 18191980.4

(51) Int. Cl. A61K 31/407; A61K 39/395; A61P 35/00

(52) U.S. Cl. CPC A61K 31/407; A61K 39/3955; A61P 35/00

(57) ABSTRACT

Methods of administering a therapeutically effective amount of Debio 1143 and a therapeutically effective amount of an anti-PD-L1 antibody or antigen-binding fragment thereof, for the treatment of cancer, are provided.

### TECHNICAL FIELD

The present invention relates to a combination product for use in the treatment of cancer. In particular, the present invention relates to combinations of (5S,8S,10aR)—N-benzhydryl-5-((S)-2-(methylamino)propanamido)-3-(3-methylbutanoyl)-6-oxodecahydropyrrolo[1,2-a][1,5]diazocine-8-carboxamide (also known as Debio 1143, AT-406, and SM-406) with immune checkpoint inhibitors for the treatment of patients with cancer.

### BACKGROUND ART

Resistance of tumor cells to apoptosis is a major problem in current cancer treatment. It is recognized that dysfunction of the apoptosis machinery is a hallmark of cancer. Defects in this machinery result in apoptosis resistance and make current anticancer therapies less effective or ineffective. The aggressive cancer cell phenotype is the result of multiple genetic and epigenetic alterations leading to deregulation of intracellular signaling pathways. Future efforts towards the development of new, molecular target-specific anticancer therapies must include new strategies that specifically target resistance of cancer cells to apoptosis.

IAPs are a class of key regulators of apoptosis characterized by the presence of one to three protein domains known as BIR. cIAP1 and cIAP2 play a critical role in the regulation of death receptor-mediated apoptosis and NFκ-B signaling pathways, which drive the expression of genes relevant for inflammation and immunity; XIAP is a central regulator of both death receptor-mediated and mitochondria-mediated apoptosis pathways. XIAP and cIAP1/2 play key roles in cancer cell resistance to a variety of anticancer drugs, and thus are promising drug targets.

Smac released from mitochondria, is an endogenous inhibitor of XIAP, cIAP1, cIAP2, and ML-IAP. Its amino-terminal tetrapeptide Ala-Val-Pro-Ile binds to a well-defined surface groove in the BIR-3 domain of XIAP. Moreover, Smac proteins can form a homodimer, interacting with both XIAP BIR-3 and BIR-2 domains to release both initiator and effector caspases to promote apoptosis.

A series of monovalent and bivalent Smac mimetics were designed and synthesized to mimic either one or two tetrapeptide Ala-Val-Pro-Ile Smac binding motifs. Both types of Smac mimetics show high binding affinities to XIAP, cIAP1/2. These Smac mimetics also show excellent activity against tumor cells, both inducing apoptosis and inhibiting cell growth and have the potential to promote anti-tumor immunity in combination with immune-oncology agents through NFκ-B signaling modulation.

Debio 1143 is a monovalent, orally available, small molecule antagonist of IAPs that has demonstrated potent single-agent anti-tumor activity in multiple models of human cancer, i.e. of the bladder, breast, head and neck, lung, ovary, pancreas, and prostate.

Immune checkpoints are regulators of immune activation. An example of such a regulator comprises programmed cell death protein 1 (PD-1) and programmed death-ligand 1 (PD-L1). PD-1 is expressed on the surface of T cells whereas PD-L1 is expressed on the surface of many more cells. Binding of PD-L1 to the PD-1 receptor inhibits TCR-mediated activation of IL-2 production and T cell proliferation.

Cancer cells have been known to overexpress PD-L1 to evade the host's immune system. Thus, PD-L1/PD-1 inhibitors have been touted as a possible therapy against cancer. Anti-PD-1 antibodies have been considered to be more promising for the treatment of cancer (You et al., 2018. J Cancer. 9(7):1200-1206). Further, recently phase III trials of Avelumab (an anti-PD-L1 antibody) for the treatment of non-small cell lung cancer (NSCLC) did not meet the prespecified endpoint of improving overall survival in patients with PD-L1 positive tumors.

Beug et al., 2014. Oncoimmunology. 3: e28541 suggests that the combination of various immunotherapies with Smac mimetics could result in effective cancer therapies. However, the combination of Debio 1143 with an anti-PD-L1 antibody is not disclosed, hinted or suggested.

WO 2016/054555 A2 discloses different combination therapies for the treatment of cancer. In some embodiments, the publication suggests combining an IAP inhibitor with an anti-PD-1 or anti-PD-L1 antibody. In particular, LCL-161 is named as a possible IAP inhibitor and it is suggested that LCL-161 should be administered once a week or once every two weeks, albeit no data is provided. Further, WO 2016/054555 A2 provides mouse model data of LCL-161 in combination with an anti-PD-1 antibody. WO 2016/054555 A2 does not disclose Debio 1143 and its combination with an anti-PD-L1 nor does WO 2016/054555 A2 provide any data where the combination of an TAP inhibitor with an anti-PD-L1 antibody is tested.

WO 2017/143449 A1 also discloses different combination therapies for the treatment of cancer. In some embodiments, the publication suggests combining an IAP inhibitor with an immune checkpoint inhibitor such as an anti-PD-1 or anti-PD-L1 antibody. Mouse model data alleging the efficacy of LCL-161 in combination with an anti-PD-1 antibody for the treatment of cancer is also provided. The combination of Debio 1143 with an anti-PD-L1 antibody is not disclosed and no data is provided for the combination of an IAP inhibitor with an anti-PD-L1 antibody.

None of the herein cited prior art documents provide any data wherein the combination of Debio 1143 with an anti-PD-L1 antibody is tested. Further, none of the prior art documents have tested the efficacy of the combination of an IAP inhibitor with an anti-PD-1 or anti-PD-L1 antibody in humans.

Therapies targeting PD-L1 and IAPs separately have shown anti-tumor effects in pre-clinical studies and in the clinic, but improving their anti-tumor efficacy and the proportion of responders remain important goals. Accordingly, there remains a need to develop novel therapeutic options for the treatment of cancers. In particular, there exists a need for methods of treating cancer that improve the efficacy of Debio 1143 or anti-PD-L1 antibodies in one or more types of cancer. The present invention provides a combination product for use in the treatment of cancer to address the above need.

### FIGURES

FIG. 1: Ex vivo T-cell activation in human peripheral blood mononuclear cells (PBMCs) upon CD3/CD28 stimulation and Debio 1143 treatment. N=1 healthy donor; values represent means of triplicates±SD.

FIG. 2: Anti-tumor activity of Debio 1143, anti-PD-1 antibody, and their combination in the subcutaneous B16F10 mouse melanoma syngeneic model. (A) Effect of Debio 1143 dose on anti-tumor efficacy of the combination. (B) Effect of the Debio 1143 dose schedule on anti-tumor efficacy of the combination.

FIG. 3: Anti-tumor activity of Debio 1143, anti-PD-L1 antibody, and their combination in the subcutaneous MBT-2 mouse bladder cancer syngeneic model.

### SUMMARY OF THE INVENTION

Without limiting the scope of the present invention, it is hypothesized that the combination of Debio 1143 with an anti-PD-L1 antibody or antigen-binding fragment thereof can target a cancer cell through the following mechanisms:
1) The anti-PD-L1 antibody or antigen-binding fragment thereof blocks the PD-1/PD-L1 axis which allows for signaling of the TCR of a CD8+ T-cell with its associated antigen presented by the cancer cell through a MHC-I molecule. Concurrent depletion of the IAPs through Debio 1143 treatment enhances T-cell activation, likely by providing a tumor necrosis factor receptor superfamily (TNFRSF) co-stimulatory response (similar to 4-1BB or OX40 activation), resulting in enhanced activation and expansion of tumor-specific CD8+ T-cells. As a result, Granzyme B (GrzB) and Perforin are secreted to kill target cells.
2) Debio 1143-mediated antagonism of the casp-3 inhibitor, XIAP, results in enhanced death of tumor cells by GrzB.
3) The depletion of cIAP1 and cIAP2 by Debio 1143 leads to increased local production of TNF-α by T-cells in the tumor microenvironment, an effect that is likely mediated by activation of the alternative NFκB pathway.
4) As a result of cIAP1/2 loss, Debio 1143-treated cancer cells are sensitized to cell death induction in the presence of proinflammatory cytokines, such as TNF-α.

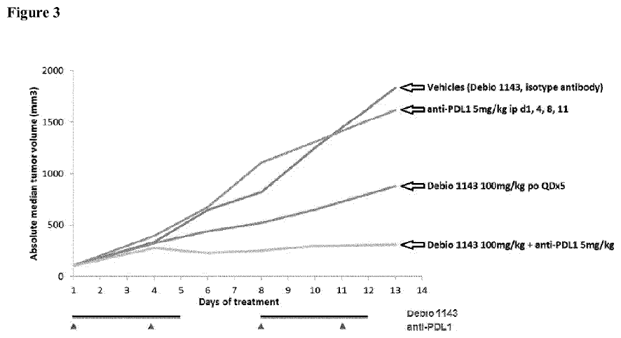

Potentiation may be additive, or it may be synergistic. The potentiating effect of the combination therapy is at least additive. The present inventors have surprisingly found that the combination of Debio 1143 with an anti-PD-L1 antibody results in an improved treatment. The inventors have shown that the potentiating effect of the combination is synergistic in a mouse model (see Example 4 and FIG. 3). Further, initial results in a clinical trial indicate that the combination therapy is effective at treating cancers such as Non-small cell lung cancer (see Example 7) and that the combination therapy is well tolerated (see Example 8).

Initial dose-dependency studies suggested that, unlike LCL-161 which is usually administered once or twice a week (WO 2016/054555 A2: p 14, lines 4-5), Debio 1143 is more effective in combination therapies when administered more frequently (see Example 3). Thus, in the ongoing clinical trials which have led to some promising results, Debio 1143 is administered for 10 consecutive days.

Avelumab and atezolizumab are also unique among currently employed anti-PD-L1 antibodies in that they are fully human IgGs with a non-mutated Fc region. Thus, avelumab comprises an antibody-dependent cellular cytotoxicity (ADCC) competent Fc region which has been shown to mediate ADCC (Boyerinas et al., 2015. Cancer Immunol Res. 3(10):1148-1157). An antibody which comprises an ADCC-competent Fc region may improve the efficacy of the present therapy by promoting ADCC lysis of the cancer cells.

Thus, the present invention provides combination products and pharmaceutical compositions, which comprise Debio 1143 and an anti-PD-L1 antibody or antigen-binding fragment thereof, that are suitable for treating cancer.

The present invention also provides methods of administering a combination product comprising Debio 1143 and anti-PD-L1. In some embodiments, the cancer is identified as PD-L1 positive cancerous disease. The anti-PD-L1 antibody and Debio 1143 can be administered in a first-line, second-line or higher treatment of the cancer. In some embodiments, the cancer is resistant to prior cancer therapy. In certain embodiments, the method is for treating a human patient having cancer comprising administering to the patient, in need thereof, a therapeutically effective amount of Debio 1143 and a therapeutically effective amount of an anti-PD-L1 antibody or antigen-binding fragment thereof. In some embodiments, the anti-PD-L1 antibody is an anti-PD-L1 IgG1 antibody. In some embodiments, the anti-PD-L1 antibody mediates antibody-dependent cell-mediated cytotoxicity (ADCC). Nevertheless, such ADCC-mediating anti-PD-L1 antibody is not toxic or does not show increased toxicity. In some embodiments, the anti-PD-L1 antibody shows cross-reactivity in mice and rhesus monkeys. In some embodiments, the anti-PD-L1 IgG1 antibody is avelumab. In some embodiments, the anti-PD-L1 antibody is administered intravenously (e.g., as an intravenous infusion) or subcutaneously. Preferably, the anti-PD-L1 antibody is administered as an intravenous infusion. More preferably, the inhibitor is administered for 50-80 minutes, most preferably as a one-hour intravenous infusion. In some embodiments, the anti-PD-L1 antibody is administered at a dose of about 10 mg/kg body weight every other week (i.e., every two weeks, or “Q2W”). In other embodiments, the anti-PD-L1 antibody and Debio 1143 are used in combination with chemotherapy (CT), radiotherapy (RT) or chemoradiotherapy (CRT).

Provided herein is also a pharmaceutical composition comprising an anti-PD-L1 antibody, Debio 1143 and at least a pharmaceutically acceptable carrier, diluent, excipient and/or adjuvant. The anti-PD-L1 antibody and Debio 1143 can be provided in a single or separate unit dosage forms. The pharmaceutical composition may be for use as a medicament, particularly for use in a method of treating cancer.

Also provided herein is an anti-PD-L1 antibody in combination with Debio 1143 for use as a medicament, particularly for use in a method of treating cancer. Similarly, Debio 1143 is provided in combination with an anti-PD-L1 antibody for use as a medicament, particularly for use in a method of treating cancer. Also provided is a combination product comprising an anti-PD-L1 antibody and Debio 1143 for any purpose, for use as a medicament or in a method of treating cancer. The combination of the anti-PD-L1 antibody and Debio 1143 can be provided in a single or separate unit dosage forms. Also provided is the use of a combination product for the manufacture of a medicament for the treatment of cancer, comprising an anti-PD-L1 antibody and Debio 1143.

Also provided is a composition comprising an anti-PD-L1 antibody for use in a method of treating cancer, wherein the composition is administered in combination with Debio 1143. Similarly, a composition comprising Debio 1143 for use in a method of treating cancer, wherein the composition is administered in combination with an anti-PD-L1 antibody is also provided. Either composition can be a pharmaceutical composition further comprising a pharmaceutically acceptable carrier, diluent, excipient and/or adjuvant.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

In some embodiments, the term “about” refers to a deviation of +10% from the recited value. When the word “about” is used herein in reference to a number, it should be understood that still another embodiment of the invention includes that number not modified by the presence of the word “about”.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient. In some embodiments, the term “administering” or “administration of” has the same meaning as understood by the person skilled in the art at the first priority date, i.e. Oct. 19, 2017, bearing in mind the skilled person's common general knowledge at the first priority date.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies). However, intact, i.e. non-fragmented, monoclonal antibodies are preferred. In some embodiments, the term “antibody” has the same meaning as understood by the person skilled in the art at the first priority date, i.e. Oct. 19, 2017, bearing in mind the skilled person's common general knowledge at the first priority date.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoictic cells is summarized in Table 3 on page 464 of Ravetch & Kinet, 1991. Annu Rev Immunol 9: 457-92.

The term “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “anti-PD-L1 antibody” or “an antibody that binds to PD-L1” refers to an antibody that is capable of specifically binding PD-L1 with sufficient affinity such that the antibody is useful as a therapeutic agent in targeting PD-L1 (e.g., avelumab). In certain embodiments, the antibody or antigen-binding fragment thereof that binds to PD-L1 includes, but is not limited to, avelumab, atezolizumab, durvalumab, and CX-072 (CytomX Therapeutics). In some embodiments, the antibody or antigen-binding fragment thereof that binds to PD-L1 is highly similar to avelumab, atezolizumab, durvalumab, or CX-072 (CytomX Therapeutics) and has no clinically meaningful differences with respect to safety and effectiveness as compared with the particular anti-PD-L1 antibody. In some embodiments, the antibody or antigen-binding fragment thereof comprises an ADCC-competent Fe region. In particular, an anti-PD-L1 antibody means an antibody that blocks binding of PD-L1 expressed on a cancer cell to PD-1. In any of the treatment method, medicaments and uses of the present invention in which a human subject is being treated, the anti-PD-L1 antibody specifically binds to human PD-L1 and blocks binding of human PD-L1 to human PD-1. The antibody may be a monoclonal antibody, human antibody, humanized antibody and/or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Examples of monoclonal antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies useful as the PD-L1 antibody in the treatment method, medicaments and uses of the present invention include, for example without limitation, avelumab (MSB0010718C), MPDL3280A (an IgG1-engineered, anti-PD-L1 antibody), BMS-936559 (a fully human, anti-PD-L1, IgG4 monoclonal antibody), MEDI4736 (an engineered IgG1 kappa monoclonal antibody with triple mutations in the Fe domain to remove antibody-dependent, cell-mediated cytotoxic activity), and an antibody which comprises the heavy chain and light chain variable regions of SEQ TD NO:24 and SEQ ID NO:21, respectively, of WO 2013/019906.

The term “cancer” refers to a group of diseases, which can be defined as any abnormal benign or malignant new growth of tissue that possesses no physiological function and arises from uncontrolled usually rapid cellular proliferation and has the potential to invade or spread to other parts of the body. Non-limiting examples include: Acute granulocytic leukemia, Acute lymphocytic leukemia, Acute myelogenous leukemia, Adenocarcinoma, Adrenal cancer, Anaplastic astrocytoma, Angiosarcoma, Appendix cancer, Astrocytoma, Basal cell carcinoma, B-Cell lymphoma, Bile duct cancer, Bladder cancer, Bone cancer, Bone marrow cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, Germ cell tumor, Glioblastoma multiforme, Glioma, Hairy cell leukemia, Head and neck cancer, Hemangioendothelioma, Hodgkin lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Mycosis Fungoides, Myelodysplastic Syndrome, Nasal cavity cancer, Nasopharyngeal cancer, Neck cancer, Neuroblastoma, Neuroendocrine tumors, Non-Hodgkin lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pituitary gland cancer, Primary central nervous system lymphoma, Prostate cancer, Rectal cancer, Renal cell carcinoma, Renal pelvis cancer, Rhabdomyosarcoma, Salivary gland cancer, Sarcoma, Bone sarcoma, Soft tissue sarcoma, Uterine sarcoma, Sinus cancer, Skin cancer, Small cell lung cancer, Small intestine cancer, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Triple-negative breast cancer, Tubal cancer, Tubular carcinoma, Urethral cancer, Uterine adenocarcinoma, Uterine cancer, Vaginal cancer and Vulvar cancer.

In a preferred embodiment, the term “cancer” refers to an advanced solid malignancy. Preferably, the cancer and/or advanced solid malignancy is selected from a group consisting of Lung Cancer, Head and Neck cancer, bladder cancer, kidney cancer, skin melanoma, colorectal cancer, ovarian cancer, breast cancer, non-Hodgkin and/or Hodgkin lymphomas. In some embodiments, the advanced solid malignancy and/or cancer is Non-Small Cell Lung Cancer. In some embodiments, the advanced solid malignancy and/or cancer is advanced or metastatic Non-Small Cell Lung Cancer. In some embodiments, the patient previously received platinum-based therapy for treatment of the advanced solid malignancy and/or cancer. In some embodiments, the patient has stage IIIB or stage IV Non-Small Cell Lung Cancer. In some embodiments, the patient with the advanced solid malignancy has histologically or cytologically confirmed stage IIIB or stage IV Non-Small Cell Lung Cancer. In some embodiments, the patient has an advanced solid malignancy that is platinum resistant, relapsed, or refractory or platinum sensitive. In some embodiments, the patient has an advanced solid malignancy that is PD-L1 positive.

The term “combination product” can refer to (i) a product comprised of two or more regulated components that are physically, chemically, or otherwise combined or mixed and produced as a single entity; (ii) two or more separate products packaged together in a single package or as a unit and comprised of drug and device products, device and biological products, or biological and drug products; (iii) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv) any investigational drug, device, or biological product packaged separately that according to its proposed labeling is for use only with another individually specified investigational drug, device, or biological product where both are required to achieve the intended use, indication, or effect.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

A “complete response” or “complete remission” or “CR” indicates the disappearance of all target lesions as defined in the RECIST v1.1 guideline or the disappearance of all signs of tumor or cancer in response to treatment. This does not always mean the cancer has been cured.

The term “Debio 1143”, “AT-406”, or “SM-406” refers to (5S,8S,10aR)—N-benzhydryl-5-((S)-2-(methylamino)propanamido)-3-(3-methylbutanoyl)-6-oxodecahydropyrrolo[1,2-a][1,5]diazocine-8-carboxamide (CAS Registry Number: 1071992-99-8) and/or pharmaceutically acceptable salts thereof. Preferably, the free base form of Debio 1143 is used in any aspect of the present invention. Its synthesis has been described previously (Cai et al., 2011. J Med Chem. 54(8):2714-26 and WO 2008/128171—Example 16).

“Disease free survival” (DFS) refers to the length of time during and after treatment that the patient remains free of disease.

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom & Winter, 1991. JMB. 227: 381; Marks et al., 1991. JMB. 222: 581). Also available for the preparation of human monoclonal antibodies are methods described in Cole et al., 1985. Monoclonal Antibodies and Cancer Therapy, Alan R. Liss, page 77; Boerner et al., 1991. J Immunol. 147(1): 86; van Dijk & van de Winkel, 2001. Curr Opin Pharmacol. 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al., 2006. PNAS USA. 103: 3557, regarding human antibodies generated via a human B-cell hybridoma technology.

“Immunoglobulin” (Ig) is used interchangeably with “antibody” herein. In some embodiments, the basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for and c isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange. Norwalk, C T, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins found in human serum: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

The terms “individual”, “patient” or “subject” are used interchangeably in the present application and are not meant to be limiting in any way. The “individual”, “patient” or “subject” can be of any age, sex and physical condition. Preferably, the methods of treatment and combination products of the present invention are for use in a human patient. In other words, the individual, patent or subject is preferably human.

“Infusion” or “infusing” refers to the introduction of a drug-containing solution into the body through a vein for therapeutic purposes. Generally, this is achieved via an intravenous bag.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

A “partial response” or “PR” refers to a decrease in the size or volume of one or more tumors or lesions, or in the extent of cancer in the body, in response to treatment. In some embodiments, a “partial response” or “PR” refers to at least a 30% decrease in the sum of diameters of target lesions, taking as reference the baseline sum diameter, in response to treatment, as defined in the RECIST v1.1 guideline.

“PD-L1 positive” cancer, including a “PD-L1 positive” cancerous disease, is one comprising cells, which have PD-L1 present at their cell surface. The term “PD-L1 positive” also refers to a cancer that produces sufficient levels of PD-L1 at the surface of cells thereof, such that an anti-PD-L1 antibody has a therapeutic effect, mediated by the binding of the said anti-PD-L1 antibody to PD-L1.

The term “pharmaceutically acceptable adjuvant” refers to any and all substances which enhance the body's immune response to an antigen. Non-limiting examples of pharmaceutically acceptable adjuvants are: Alum, Freund's Incomplete Adjuvant, MF59, synthetic analogs of dsRNA such as poly(I:C), bacterial LPS, bacterial flagellin, imidazolquinolines, oligodeoxynucleotides containing specific CpG motifs, fragments of bacterial cell walls such as muramyl dipeptide and Quil-A®.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition. Pharmaceutical compositions comprising Debio 1143 preferably comprise Starch 1500 (reference to quality standard: Ph. Eur. 01/2010:1267) as a pharmaceutically acceptable excipient.

“Platinum-based therapy” refers to any therapy which involves the use of platinum-based agents, such as cisplatin, carboplatin and oxaliplatin for the treatment of cancer. Platinum-based agents are alkylating agents which bind covalently to DNA and cross-links DNA strands, resulting in inhibition of DNA synthesis and function as well as inhibition of transcription. Platinum-based chemotherapy combinations have demonstrated improvements over single-agent therapy in advanced NSCLC (see Dubey & Schiller, 2004. Hematol Oncol Clin N Am. 18:101-114). Thus, in some embodiments, the platinum-based therapy is a platinum-based doublet chemotherapy (Du & Morgensztern, 2015. Cancer J. 21(5):366-370). According to current guidelines, the first-line treatment strategy for advanced NSCLC should take into account age, histology, molecular pathology, comorbidities, and the performance status of patients, and platinum-based doublet chemotherapy (PT-DC) has been recommended as the standard first-line treatment for such individuals, especially those without epidermal growth factor receptor (EGFR) mutations (Hu et al., 2016. Medicine (Baltimore). 95(28):e4183).

The term “platinum-based therapy cycle” refers to a course of treatment that is repeated on a regular schedule with periods of rest in between. For example, treatment given for one week followed by three weeks of rest is one treatment cycle.

“Progressive disease” or “disease that has progressed” refers to the appearance of one more new lesions or tumors and/or the unequivocal progression of existing non-target lesions, preferably, as defined in the RECIST v1.1 guideline. Progressive disease or disease that has progressed can also refer to a tumor growth of more than 20 percent since treatment began, either due to an increase in mass or in spread of the tumor.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

The term “RECIST” means Response Evaluation Criteria in Solid Tumours. RECIST guideline, criteria, or standard, describes a standard approach to solid tumor measurement and definitions for objective assessment of change in tumor size for use in adult and pediatric cancer clinical trials. RECIST v1.1 means version 1.1 of the revised RECIST guideline and it is published in European Journal of Cancers 45 (2009) 228-247.

The term “respond favorably” generally refers to causing a beneficial state in a subject. With respect to cancer treatment, the term refers to providing a therapeutic effect on the subject. Positive therapeutic effects in cancer can be measured in a number of ways (See, Weber, 2009. J Nucl Med. 50 Suppl 1:1S-10S). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof.

“Stable disease” refers to disease without progression or relapse, preferably, as defined in the RECIST v1.1 guideline. In stable disease there is neither sufficient tumor shrinkage to qualify for partial response, nor sufficient tumor increase to qualify as progressive disease.

The term “therapeutically effective amount” refers to an amount of Debio 1143, and/or antibody or antigen-binding fragment thereof which has a therapeutic effect and which is able to treat cancer. In the case of cancer, e.g., an advanced solid malignancy, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

“Time to Tumor Progression” (TTP) is defined as the time from enrollment to disease progression. TTP is generally measured using the RECIST v1.1 criteria.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Alternatively, the terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

PFS, DFS, and OS can be measured by standards set by the National Cancer Institute and the U.S. Food and Drug Administration for the approval of new drugs. See Johnson et al., (2003) J. Clin. Oncol. 21(7):1404-1411.

### Methods of Use and Pharmaceutical Compositions

The present invention provides a combination product comprising Debio 1143 and an anti-PD-L1 antibody or antigen-binding fragment thereof for use in a method of treating cancer.

The present invention also provides a composition comprising Debio 1143 for use in a method of treating cancer comprising administering an anti-PD-L1 antibody or antigen-binding fragment thereof. Alternatively, the present invention provides an anti-PD-L1 antibody or antigen-binding fragment thereof for use in a method of treating cancer comprising administering Debio 1143.

The present invention also provides methods of administering a combination product comprising Debio 1143 and an anti-PD-L1 antibody or antigen-binding fragment thereof. Further, the present invention provides methods of administering Debio 1143 and an anti-PD-L1 antibody or antigen-binding fragment thereof. In certain embodiments, the method is for treating a human patient having cancer comprising administering to the patient, in need thereof, a therapeutically effective amount of Debio 1143 and a therapeutically effective amount of an anti-PD-L1 antibody. In some embodiments, the anti-PD-L1 antibody is an anti-PD-L1 IgG1 antibody. In some embodiments, the anti-PD-L1 IgG1 antibody is avelumab.

In certain embodiments, the method for treating cancer is a method for treating a human patient having cancer comprising administering to the patient, in need thereof, a therapeutically effective amount of Debio 1143 and a therapeutically effective amount of an anti-PD-L1 antibody. In some embodiments, the anti-PD-L1 antibody is an anti-PD-L1 IgG1 antibody. In some embodiments, the anti-PD-L1 IgG1 antibody is avelumab.

In some embodiments, the therapeutically effective amount of Debio 1143 is about 75 to about 250 mg per day. Preferably, the therapeutically effective amount of Debio 1143 is about 75-100, 75-125, 75-150, 75-175, 75-200, 75-225, 100-125, 100-150, 100-175, 100-200, 100-225, 125-150, 125-175, 125-200, 125-225, 150-175, 150-200, 150-225, 175-200, 175-225 or 200-225 mg per day. In some embodiments, the therapeutically effective amount of Debio 1143 is about 75, 100, 125, 150, 175, 200, 225 or 250 mg per day.

In some embodiments, the Debio 1143 is administered orally. In some embodiments, the Debio 1143 is administered in capsular form or tablet form. In some embodiments, the Debio 1143 is administered orally as a capsule containing 75, 100, 125, 150, 175, 200, 225 or 250 mg Debio 1143. In some embodiments, the Debio 1143 is administered orally as a tablet containing 75, 100, 125, 150, 175, 200, 225 or 250 mg Debio 1143.

In certain embodiments, the therapeutically effective amount of Debio 1143 is administered as one dose one time per day. In certain embodiments, the therapeutically effective amount of Debio 1143 is divided into multiple doses that are administered as multiple doses two, three, or four times per day.

In some embodiments, the Debio 1143 is administered daily for 10 consecutive days. In some embodiments, the Debio 1143 is administered once daily for 10 consecutive days. In some embodiments, the method of treatment comprises a 28 day cycle comprising administering the Debio 1143 for 10 consecutive days, followed by administering no Debio 1143 for 4 consecutive days.

In one embodiment, the anti-PD-L1 antibody is a monoclonal antibody. In one embodiment, the anti-PD-L1 antibody exerts antibody-dependent cell-mediated cytotoxicity (ADCC). In one embodiment, the anti-PD-L1 antibody is a human or humanized antibody. In various embodiments, the anti-PD-L1 antibody is characterized by a combination of one or more of the foregoing features, as defined above.

In some embodiments, the anti-PD-L1 antibody is an anti-PD-L1 IgG antibody. In some embodiments, the anti-PD-L1 IgG antibody is selected from the group consisting of avelumab, atezolizumab, durvalumab, and CX-072 (CytomX Therapeutics). In some embodiments, the anti-PD-L1 antibody is avelumab (marketed in the United States under the Tradename Bavencio®). Avelumab is disclosed in International Patent Publication No. WO 2013/079174, the disclosure of which is hereby incorporated by reference in its entirety. Avelumab (formerly designated MSB0010718C) is a fully human monoclonal antibody of the immunoglobulin (Ig) G1 isotype (see e.g., WO 2013/079174). Avelumab selectively binds to PD-L1 and competitively blocks its interaction with PD-1. The mechanisms of action rely on the inhibition of PD-1/PD-L1 interaction and on natural killer (NK)-based ADCC (see e.g., Boyerinas et al, 2015. Cancer Immunol Res. 3: 1148). Compared with anti-PD-1 antibodies that target T cells, avelumab targets tumor cells and therefore, it is expected to have fewer side effects, including a lower risk of autoimmune-related safety issues, as the blockade of PD-L1 leaves the PD-L2/PD-1 pathway intact to promote peripheral self-tolerance (see e.g., Latchman et al., 2001. Nat Immunol. 2(3): 261).

In certain embodiments, a therapeutically effective amount of an anti-PD-L1 antibody (e.g. avelumab), or antigen-binding fragment thereof, is administered in the methods. The therapeutically effective amount is sufficient for treating one or more symptoms of a disease or disorder associated with PD-L1 and IAP, respectively. In some embodiments that employ an anti-PD-L1 antibody in the combination therapy, the dosing regimen will comprise administering the anti-PD-L1 antibody at a dose of about 1, 2, 3, 4, 5, 6, 7, 8, 9, 10, 11, 12, 13, 14, 15, 16, 17, 18, 19 or 20 mg/kg at intervals of about 14 days (+2 days) or about 21 days (+2 days) or about 30 days (±2 days) throughout the course of treatment. In other embodiments that employ an anti-PD-L1 antibody in the combination therapy, the dosing regimen will comprise administering the anti-PD-L1 antibody at a dose of from about 0.005 mg/kg to about 10 mg/kg, with intra-patient dose escalation. In certain embodiments, the therapeutically effective amount of anti-PD-L1 antibody (e.g. avelumab), or antigen-binding fragment thereof, is about 10 mg/kg. In some embodiments, the anti-PD-L1 antibody (e.g., avelumab), antigen-binding fragment thereof, is administered intravenously. In some embodiments, the anti-PD-L1 antibody is avelumab and the therapeutically effective amount of avelumab is about 10 mg/kg. In some embodiments, the avelumab is administered once every two weeks. In some embodiments, the avelumab is administered on days 1 and 15 of a 28-day cycle. In some embodiments, avelumab is administered intravenously. In certain embodiments, the anti-PD-L1 antibody is administered intravenously for 50-80 minutes at a dose of about 10 mg/kg body weight every two weeks. In a more preferred embodiment, the avelumab dose will be 10 mg/kg body weight administered as 1-hour intravenous infusions every 2 weeks (Q2W). Given the variability of infusion pumps from site to site, a time window of minus 10 minutes and plus 20 minutes is permitted. Pharmacokinetic studies demonstrated that the 10 mg/kg dose of avelumab achieves excellent receptor occupancy with a predictable pharmacokinetics profile (see e.g., Heery et al., 2015. Proc ASCO Annual Meeting: abstract 3055). This dose is well tolerated, and signs of antitumor activity, including durable responses, have been observed. Avelumab may be administered up to 3 days before or after the scheduled day of administration of each cycle due to administrative reasons.

In some embodiments, the method further comprises administering an antihistamine (anti-H1) and acetaminophen to the patient prior to administering the anti-PD-L1 antibody or antigen-binding fragment thereof. In some embodiments, the antihistamine (anti-H1) and acetaminophen are administered to the patient about 30 minutes to about 60 minutes prior administering the anti-PD-L1 antibody or antigen-binding fragment thereof. In some embodiments, the antihistamine (anti-H1) and acetaminophen are administered prior to each of the first four administrations of anti-PD-L1 antibody or antigen-binding fragment thereof. In some embodiments, the antihistamine (anti-H1) is diphenhydramine. In some embodiments, about 25 to about 50 mg diphenhydramine is administered in the method.

In various embodiments, the method of the invention is employed as a first, second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

The safety and the clinical benefit offered by the therapeutic combination of the invention warrants a first-line setting in cancer patients. Particularly, the combination may become a new standard treatment for patients suffering from a cancer. In another embodiment of the invention, the therapeutic combination of the invention is applied in a later line of treatment, particularly a second-line or higher treatment of the cancer. There is no limitation to the prior number of therapies provided that the subject underwent at least one round of prior cancer therapy. The round of prior cancer therapy refers to a defined schedule/phase for treating a subject with, e.g., one or more immunotherapeutic agents (e.g., an anti-PD-L1 antibody), chemotherapeutic agents, radiotherapy or chemoradiotherapy, and the subject failed with such previous treatment, which was either completed or terminated ahead of schedule. One reason could be that the cancer was resistant to prior therapy. The addition of Debio 1143 will suppress this mechanism of resistance and restore the effect of the immunotherapy. The set of patients with resistance becomes treatable and show improved responses.

As the mode of action of Debio 1143 is different from that of the anti-PD-L1 antibodies, the chances to have enhanced immune-related adverse events (irAE) are small although both agents are targeting the immune system. The absence of overlapping immune features in nonclinical findings or in published clinical results makes the risk low for the combination therapy of the invention to show enhanced adverse events above what is generally observed in the class of PD-L1 targeting agents. The identified and potential risks for the anti-PD-L1 antibody of the invention, preferably avelumab, and for Debio 1143 of the invention, in each case as single agent, are considered to represent the potential risks for the combination treatment as well.

The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of an anti-PD-L1 antibody/Debio 1143 combination seems to be much better than the SoC chemotherapy. In one embodiment, an anti-PD-L1 antibody/Debio 1143 combination may be as effective and better tolerated than SoC chemotherapy in patients with cancer resistant to mono- and/or poly-chemotherapy, radiotherapy or chemoradiotherapy.

In certain embodiments, methods of treating a human patient having Non-Small Cell Lung Cancer comprising administering to the patient, in need thereof, about 75 mg to about 250 mg of Debio 1143 and about 10 mg/kg avelumab are provided herein.

In certain embodiments, methods of treating a human patient having advanced or metastatic Non-Small Cell Lung Cancer comprising administering to the patient, in need thereof, about 75 mg to about 250 mg of Debio 1143 and about 10 mg/kg avelumab are provided herein. In some embodiments, the patient with advanced or metastatic Non-Small Cell Lung Cancer previously received platinum-based therapy for the Non-Small Cell Lung Cancer. In some embodiments, the patient is orally administered the Debio 1143. In some embodiments, the Debio 1143 is provided in capsular form. In some embodiments, the patient is orally administered the Debio 1143 for 10 consecutive days.

In some embodiments, the method of treatment comprises a 28 day cycle comprising
(a) administering the Debio 1143 for 10 consecutive days; and
(b) administering no Debio 1143 for 4 consecutive days.

In some embodiments, the method of treatment comprises administering the Debio 1143 for 10 consecutive days followed by 4 consecutive days wherein the Debio 1143 is not administered.

Debio 1143 is more effective in combination therapies when administered more frequently (see Example 3). Thus, administering Debio 1143 for 10 consecutive days should be more effective than administering Debio 1143 less frequently, for example, once or twice a week. Further, the four consecutive days in which no Debio 1143 is administered follows the ten consecutive days of treatment to ensure that the patient can recover from the treatment.

In some embodiments, the avelumab is administered once every two weeks. In some embodiments, the avelumab is administered on days 1 and 15 of a 28-day cycle. In some embodiments, the avelumab is administered intravenously. In some embodiments, the method comprises administering an antihistamine (anti-H1) and acetaminophen to the patient prior to administering the avelumab. In some embodiments, the antihistamine (anti-H1) and acetaminophen are administered to the patient about 30 minutes to about 60 minutes prior to administering the avelumab. In some embodiments, the antihistamine (anti-H1) and acetaminophen are administered prior to each of the first four administrations of avelumab. In some embodiments, the antihistamine (anti-H1) is diphenhydramine.

In some embodiments, about 25 to about 50 mg diphenhydramine is administered.

In certain embodiments, methods of treatment comprising a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period; and
(d) administering no Debio 1143 for a second 4 consecutive day period
   are provided herein. In some embodiments, the avelumab is administered once every two weeks. In some embodiments, the avelumab is administered on days 1 and 15 of the 28-day cycle. In some embodiments, the avelumab is administered intravenously. In some embodiments, the method further comprises administering an antihistamine (anti-H1) and acetaminophen to the patient prior to administering the avelumab. In some embodiments, the antihistamine (anti-H1) and acetaminophen are administered to the patient about 30 minutes to about 60 minutes prior to administering the avelumab. In some embodiments, the antihistamine (anti-H1) and acetaminophen are administered prior to each of the first four administrations of avelumab. In some embodiments, the antihistamine (anti-H1) is diphenhydramine. In some embodiments, about 25 to about 50 mg diphenhydramine is administered. In some embodiments, the patient previously received platinum-based therapy for treatment of the Non-Small Cell Lung Cancer.

In certain embodiments, the method of treatment comprises a 28-day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle.

In certain embodiments, methods of treating a human patient having Non-Small Cell Lung Cancer comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein.

By following the above treatment protocol, an effective treatment of NSCLC has been achieved (see Example 6).

In certain embodiments, methods of treating a human patient having advanced or metastatic Non-Small Cell Lung Cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein. In some embodiments, the Debio 1143 is administered in capsular form.

In certain embodiments, methods of treating a human patient having Non-Small Cell Lung Cancer comprising administering to the patient, in need thereof, about 75 mg to about 250 mg of Debio 1143 and about 10 mg/kg avelumab are provided herein, wherein the methods of treatment results in a decrease in size of at least 10% of a cancer-associated lesion compared to the size of the lesion before the start of the method of treatment.

In certain embodiments, methods of treating a human patient having advanced or metastatic Non-Small Cell Lung Cancer comprising administering to the patient, in need thereof, about 75 mg to about 250 mg of Debio 1143 and about 10 mg/kg avelumab wherein the methods of treatment results in a decrease in size of at least 10% of a cancer-associated lesion compared to the size of the lesion before the start of the method of treatment are provided herein. In some embodiments, the patient with advanced or metastatic Non-Small Cell Lung Cancer previously received platinum-based therapy for the Non-Small Cell Lung Cancer. In some embodiments, the patient is orally administered the Debio 1143. In some embodiments, the Debio 1143 is provided in capsular form. In some embodiments, the patient is orally administered the Debio 1143 for 10 consecutive days.

In certain embodiments, methods of treating a human patient having Non-Small Cell Lung Cancer comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle;
   wherein the methods of treatment results in a decrease in size of at least 10% of a cancer-associated lesion compared to the size of the lesion before the start of the method of treatment are provided herein.

In certain embodiments, methods of treating a human patient having advanced or metastatic Non-Small Cell Lung Cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle;
   wherein the methods of treatment results in a decrease in size of at least 10% of a cancer-associated lesion compared to the size of the lesion before the start of the method of treatment are provided herein. In some embodiments, the Debio 1143 is administered in capsular form.

In certain embodiments, methods of treating a human patient having Non-Small Cell Lung Cancer comprising administering to the patient, in need thereof, about 75 mg to about 250 mg of Debio 1143 and about 10 mg/kg avelumab are provided herein, wherein the methods of treatment results in a decrease in size of at least 20% of a cancer-associated lesion compared to the size of the lesion before the start of the method of treatment.

In certain embodiments, methods of treating a human patient having advanced or metastatic Non-Small Cell Lung Cancer comprising administering to the patient, in need thereof, about 75 mg to about 250 mg of Debio 1143 and about 10 mg/kg avelumab wherein the methods of treatment results in a decrease in size of at least 20% of a cancer-associated lesion compared to the size of the lesion before the start of the method of treatment are provided herein. In some embodiments, the patient with advanced or metastatic Non-Small Cell Lung Cancer previously received platinum-based therapy for the Non-Small Cell Lung Cancer. In some embodiments, the patient is orally administered the Debio 1143. In some embodiments, the Debio 1143 is provided in capsular form. In some embodiments, the patient is orally administered the Debio 1143 for 10 consecutive days.

In certain embodiments, methods of treating a human patient having Non-Small Cell Lung Cancer comprising orally administering to the patient, in need thereof, about 75 mg to about 250 mug of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle;
   wherein the methods of treatment results in a decrease in size of at least 20% of a cancer-associated lesion compared to the size of the lesion before the start of the method of treatment are provided herein.

In certain embodiments, methods of treating a human patient having advanced or metastatic Non-Small Cell Lung Cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle;
   wherein the methods of treatment results in a decrease in size of at least 20% of a cancer-associated lesion compared to the size of the lesion before the start of the method of treatment are provided herein. In some embodiments, the Debio 1143 is administered in capsular form.

In certain embodiments, methods of treating a human patient having Non-Small Cell Lung Cancer comprising administering to the patient, in need thereof, about 75 mg to about 250 mg of Debio 1143 and about 10 mg/kg avelumab are provided herein, wherein the methods of treatment results in a decrease in size of at least 30% of a cancer-associated lesion compared to the size of the lesion before the start of the method of treatment.

In certain embodiments, methods of treating a human patient having advanced or metastatic Non-Small Cell Lung Cancer comprising administering to the patient, in need thereof, about 75 mg to about 250 mg of Debio 1143 and about 10 mg/kg avelumab wherein the methods of treatment results in a decrease in size of at least 30% of a cancer-associated lesion compared to the size of the lesion before the start of the method of treatment are provided herein. In some embodiments, the patient with advanced or metastatic Non-Small Cell Lung Cancer previously received platinum-based therapy for the Non-Small Cell Lung Cancer. In some embodiments, the patient is orally administered the Debio 1143. In some embodiments, the Debio 1143 is provided in capsular form. In some embodiments, the patient is orally administered the Debio 1143 for 10 consecutive days.

In certain embodiments, methods of treating a human patient having Non-Small Cell Lung Cancer comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle;
   wherein the methods of treatment results in a decrease in size of at least 30% of a cancer-associated lesion compared to the size of the lesion before the start of the method of treatment are provided herein.

In certain embodiments, methods of treating a human patient having advanced or metastatic Non-Small Cell Lung Cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(c) administering the avelumab on day 1 of the 28-day cycle; and
(f) administering the avelumab on day 15 of the 28-day cycle;
   wherein the methods of treatment results in a decrease in size of at least 30% of a cancer-associated lesion compared to the size of the lesion before the start of the method of treatment are provided herein. In some embodiments, the Debio 1143 is administered in capsular form.

In certain embodiments, methods of treating a human patient having Bladder cancer comprising administering to the patient, in need thereof, about 75 mg to about 250 mg of Debio 1143 and about 10 mg/kg avelumab are provided herein.

In certain embodiments, methods of treating a human patient having advanced or metastatic Bladder cancer comprising administering to the patient, in need thereof, about 75 mg to about 250 mg of Debio 1143 and about 10 mg/kg avelumab are provided herein. In some embodiments, the patient with advanced or metastatic Bladder cancer previously received platinum-based therapy for the Bladder cancer. In some embodiments, the patient is orally administered the Debio 1143. In some embodiments, the Debio 1143 is provided in capsular form. In some embodiments, the patient is orally administered the Debio 1143 for 10 consecutive days.

In certain embodiments, methods of treating a human patient having Bladder cancer comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein.

In certain embodiments, methods of treating a human patient having advanced or metastatic Bladder cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein. In some embodiments, the Debio 1143 is administered in capsular form.

In certain embodiments, methods of treating a human patient having skin melanoma comprising administering to the patient, in need thereof, about 75 mg to about 250 mg of Debio 1143 and about 10 mg/kg avelumab are provided herein.

In certain embodiments, methods of treating a human patient having advanced or metastatic skin melanoma comprising administering to the patient, in need thereof, about 75 mg to about 250 mg of Debio 1143 and about 10 mg/kg avelumab are provided herein. In some embodiments, the patient with advanced or metastatic skin melanoma previously received platinum-based therapy for the skin melanoma. In some embodiments, the patient is orally administered the Debio 1143. In some embodiments, the Debio 1143 is provided in capsular form. In some embodiments, the patient is orally administered the Debio 1143 for 10 consecutive days.

In certain embodiments, methods of treating a human patient having skin melanoma comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein.

In certain embodiments, methods of treating a human patient having advanced or metastatic skin melanoma after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle
   are provided herein. In some embodiments, the Debio 1143 is administered in capsular form.

In certain embodiments, in addition to administering the Debio 1143 and the anti-PD-L1 antibody (e.g., avelumab), or antigen-binding fragment thereof, the method of treatment further comprises administering an antihistamine (anti-H1) (e.g., diphenhydramine) and/or acetaminophen to the patient.

In some embodiments, the method further comprises administering an antihistamine (anti-H1) to the patient prior to administering the avelumab. In certain embodiments, the method further comprises administering acetaminophen to the patient prior to administering the avelumab. In some embodiments, the method further comprises administering an antihistamine (anti-H1) and acetaminophen to the patient prior to administering the avelumab. In certain embodiments, the antihistamine (anti-H1) is administered to the patient about 30 minutes to about 60 minutes prior to administering the avelumab. In certain embodiments, the acetaminophen is administered to the patient about 30 minutes to about 60 minutes prior to administering the avelumab. In certain embodiments, the antihistamine (anti-H1) and acetaminophen are administered to the patient about 30 minutes to about 60 minutes prior to administering the avelumab. In certain embodiments, the antihistamine (anti-H1) is diphenhydramine. In certain embodiments, about 25 to about 50 mg diphenhydramine is administered. In certain embodiments, the therapeutically effective amount of Debio 1143 is administered as one dose one time per day. In certain embodiments, the therapeutically effective amount of Debio 1143 is divided into multiple doses that are administered as multiple doses two, three, or four times per day.

In some embodiments, the platinum-based therapy comprised administering one of more platinum-based agents selected from the group consisting of cisplatin, carboplatin, and oxaliplatin. In some embodiments, the patient has relapsed or progressed after being administered the platinum-based therapy but before being administered the Debio 1143. In some embodiments, the patient previously underwent at least one platinum-based therapy cycle. In some embodiments, the patient previously underwent at least two, three, four, five or six platinum-based therapy cycles. In some embodiments, the platinum-based therapy was stopped after at least one cycle because the disease progressed despite the platinum-based therapy. In some embodiments, the platinum-based therapy was stopped after at least one cycle due to toxicity, wherein the toxicity is associated with the platinum-based therapy.

In one embodiment, the cancer is identified as a PD-L1-positive cancerous disease. Pharmacodynamic analyses show that tumor expression of PD-L1 might be predictive of treatment efficacy. According to the invention, the cancer is preferably considered to be PD-L1 positive if between at least 0.1% and at least 10% of the cells of the cancer have PD-L1 present at their cell surface, more preferably between at least 0.5% and 5%, most preferably at least 1%.

In some embodiments, the therapeutically effective amount of Debio 1143 and the anti-PD-L1 antibody (e.g. avelumab) or antigen-binding fragment thereof is administered to a patient with an increased expression level of PD-L1. In some embodiments, the PD-L1 expression level is measured by immunohistochemistry (IHC). Immunohistochemistry with anti-PD-L1 primary antibodies can be performed on serial cuts of formalin fixed and paraffin embedded specimens from patients treated with an anti-PD-L1 antibody, such as avelumab, and Debio 1143. In some embodiments, at least 1% of the cells exhibit PD-L1 expression. Preferably, at least 1% of the cancer cells exhibit PD-L1 expression.

This disclosure also provides a kit for determining if the combination of the invention is suitable for therapeutic treatment of a cancer patient, comprising means for determining a protein level of PD-L1, or the expression level of its RNA, in a sample isolated from the patient and instructions for use. In another aspect, the kit further comprises avelumab for immunotherapy or Debio 1143. In one aspect of the invention, the determination of a high PD-L1 level indicates increased PFS or OS when the patient is treated with the therapeutic combination of the invention. In one embodiment of the kit, the means for determining the PD-L1 peptide level are antibodies with specific binding to PD-L1, respectively.

In some embodiments, the combination product is a pharmaceutical combination product and further comprises a pharmaceutically acceptable carrier, diluent, excipient and/or adjuvant. In some embodiments, the anti-PD-L1 antibody or antigen-binding fragment thereof and/or Debio 1143 is comprised within one or more pharmaceutical compositions further comprising a pharmaceutically acceptable carrier, diluent, excipient and/or adjuvant.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

In some embodiments, the method of treatment results in a decrease of at least one grade of the Eastern Cooperative Oncology Group Performance Status (ECOG-PS) scale in comparison to the ECOG-PS grade before the start of the method of treatment if the grade before the start of the method of treatment is 4 or less, preferably 2 or less. The response criteria for the ECOG-PS scale are well known in the art (see Oken et al., 1982. Am J Clin Oncol. 5(6):649-55).

In some embodiments, the method of treatment results in a decrease in size of a cancer-associated lesion compared to the size of the lesion before the start of the method of treatment. In some embodiments, the method of treatment results in a decrease in size of at least 10, 20 or 30% of a cancer-associated lesion compared to the size of the lesion before the start of the method of treatment. The size of the lesion can be determined by performing a computed tomography (CT) scan of the patient.

In a further aspect, the anti-PD-L1 antibody and Debio 1143 are administered sequentially in either order or substantially simultaneously. In some embodiments, the combination regimen comprises the steps of: (a) under the direction or control of a physician, the subject receiving the PD-L1 antibody prior to first receipt of Debio 1143; and (b) under the direction or control of a physician, the subject receiving Debio 1143. In some embodiments, the combination regimen comprises the steps of: (a) under the direction or control of a physician, the subject receiving Debio 1143 prior to first receipt of the PD-L1 antibody; and (b) under the direction or control of a physician, the subject receiving the PD-L1 antibody. In some embodiments, the combination regimen comprises the steps of: (a) prescribing the subject to self-administer, and verifying that the subject has self-administered, the PD-L1 antibody prior to first administration of Debio 1143; and (b) administering Debio 1143 to the subject. In some embodiments, the combination regimen comprises the steps of: (a) prescribing the subject to self-administer, and verifying that the subject has self-administered, Debio 1143 prior to first administration of the PD-L1 antibody; and (b) administering the PD-L1 antibody to the subject. In some embodiments, the combination regimen comprises, after the subject has received the PD-L1 antibody prior to the first administration of Debio 1143, administering Debio 1143 to the subject. In some embodiments, the combination regimen comprises, after the subject has received Debio 1143 prior to first administration of the anti-PD-L1 antibody, administering the anti-PD-L1 antibody to the subject.

Also provided herein is an anti-PD-L1 antibody for use as a medicament in combination with Debio 1143. Similarly provided is Debio 1143 for use as a medicament in combination with an anti-PD-L1 antibody. Also provided is an anti-PD-L1 antibody for use in the treatment of cancer in combination with Debio 1143. Similarly provided is Debio 1143 for use in the treatment of cancer in combination with an anti-PD-L1 antibody. Also provided is a combination product comprising an anti-PD-L1 antibody and Debio 1143 for use as a medicament. Also provided is the use of a combination product for the manufacture of a medicament for the treatment of cancer, comprising an anti-PD-L1 antibody and Debio 1143. The aforementioned combinations and combination products are provided in a single or separate unit dosage forms.

In a further aspect, the invention relates to a kit comprising an anti-PD-L1 antibody and a package insert comprising instructions for using the anti-PD-L1 antibody in combination with Debio 1143 to treat or delay progression of a cancer in a subject. Also provided is a kit comprising Debio 1143 and a package insert comprising instructions for using Debio 1143 in combination with an anti-PD-L1 antibody to treat or delay progression of a cancer in a subject. Also provided is a kit comprising an anti-PD-L1 antibody and Debio 1143, and a package insert comprising instructions for using the anti-PD-L1 antibody and Debio 1143 to treat or delay progression of a cancer in a subject. The kit can comprise a first container, a second container and a package insert, wherein the first container comprises at least one dose of a medicament comprising an anti-PD-L1 antibody, the second container comprises at least one dose of a medicament comprising Debio 1143, and the package insert comprises instructions for treating a subject for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1 expression.

### Anti-PD-L1 Antibodies

The term “antibody” includes intact molecules. Constant regions of the antibodies can be altered, e.g., mutated, to modify the properties of the antibody (e.g., to increase or decrease one or more of: Fc receptor binding, antibody glycosylation, the number of cysteine residues, effector cell function or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat et al., 1991. Sequences of Proteins of Immunological Interest, Fifth Edition, U.S. Department of Health and Human Services, NIH Publication No. 91-3242; Chothia et al., 1987. J Mol Biol. 196:901-917; and the AbM definition used by Oxford Molecular's AbM antibody modeling software). See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The precise amino acid sequence boundaries of a given CDR can be determined using any of a number of well-known schemes, including those described by Kabat et al., 1991. “Sequences of Proteins of Immunological Interest,” 5th Ed. Public Health Service, National Institutes of Health, Bethesda, MD (“Kabat” numbering scheme), Al-Lazikani et al., 1997. JMB. 273:927-948 (“Chothia” numbering scheme). As used herein, the CDRs defined according the “Chothia” number scheme are also sometimes referred to as “hypervariable loops”.

The terms “monoclonal antibody” as used herein refers to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody or antigen-binding fragment thereof can be a polyclonal or a monoclonal antibody. In other embodiments, the antibody can be recombinantly produced, e.g., produced by phage display or by combinatorial methods. Preferably, the antibody or antigen-binding fragment thereof is a monoclonal antibody or antigen-binding fragment thereof.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. 1991. Bio/Technology. 9:1370-1372; Hay et al., 1992. Hum Antibod Hybridomas. 3:81-85; Huse et al., 1989. Science. 246:1275-1281; Griffths et al., 1993. EMBO J. 12:725-734; Hawkins et al., 1992. J Mol Biol. 226:889-896; Clackson et al., 1991. Nature. 352:624-628; Gram et al., 1992. PNAS. 89:3576-3580; Garrad et al., 1991. Bio/Technology. 9:1373-1377; Hoogenboom et al., 1991. Nuc Acid Res. 19:4133-4137; and Barbas et al., 1991. PNAS. 88:7978-7982, the contents of all of which are incorporated by reference herein).

In one embodiment, the antibody is a fully human antibody (e.g., an antibody made in a mouse which has been genetically engineered to produce an antibody from a human inmmunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, et al., 1994. Nature. 368:856-859; Green, et al., 1994. Nature Genet. 7:13-21; Morrison et al., 1994 Proc Natl Acad Sci USA. 81:6851-6855; Bruggeman et al., 1993. Year Immunol. 7:33-40; Tuaillon et al., 1993. PNAS. 90:3720-3724; Bruggeman et al., 1991. Eur J Immunol. 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., 1988. Science. 240:1041-1043; Liu et al., 1987. PNAS. 84:3439-3443; Liu et al., 1987. J Immunol. 139:3521-3526; Sun et al., 1987. PNAS. 84:214-218; Nishimura et al., 1987. Canc Res. 47:999-1005; Wood et al., 1985. Nature. 314:446-449; and Shaw et al., 1988. J Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to anti-PD-L1. Preferably, the donor will be a rodent antibody, e.g., a rat or mouse antibody, and the recipient will be a human framework or a human consensus framework. Typically, the inmmunoglobulin providing the CDRs is called the “donor” and the immunoglobulin providing the framework is called the “acceptor”. In one embodiment, the donor immunoglobulin is a non-human (e.g., rodent) immunoglobulin. The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (see e.g., Winnaker, From Genes to Clones (Verlagsgesellschaft, Weinheim, Germany 1987)). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, 1985. Science. 229:1202-1207; Oi et al., 1986. BioTechniques. 4:214; Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al., 1986. Nature. 321:552-525; Verhoeyan et al., 1988. Science. 239:1534; Beidler et al., 1988. J Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody can be a single chain antibody. A single-chain antibody (scFV) may be engineered (see, for example, Colcher et al., 1999. Ann N Y Acad Sci. 880:263-80; and Reiter, 1996. Clin Cancer Res. 2:245-52). The single chain antibody can be dimerized or multimerized to generate multivalent antibodies having specificities for different epitopes of the same target protein.

In yet other embodiments, the antibody has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody has a light chain constant region chosen from, e.g., the (e.g., human) light chain constant regions of kappa or lambda. The constant region can be altered, e.g., mutated, to modify the properties of the antibody (e.g., to increase or decrease one or more of: Fe receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fe receptor binding region.

Methods for altering an antibody constant region are known in the art. Antibodies with altered function, e.g. altered affinity for an effector ligand, such as FcR on a cell, or the Cl component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Avelumab is marketed under the Tradename Bavencio®. Atezolizumab is marketed under the Tradename Tecentriq®. Durvalumab is marketed under the Tradename Imfinzi™. CX-072 is currently being investigated in clinical trials.

A full-length amino acid sequence for PD-L1 is provided at UniProtKB Accession No. Q15116 and herein as SEQ ID NO: 1:

(SEQ ID NO: 1)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL,
the signal sequence of which is
(SEQ ID NO: 2)
MQIPQAPWPVVWAVLQLGWR.

Thus, in some embodiments, an anti-PD-L1 antibody or antigen-binding fragment thereof specifically binds to an epitope in SEQ ID NO: 1 or to an epitope in the mature version of SEQ ID NO: 1 (i.e., SEQ ID NO: 1 lacking the signal sequence).

In some embodiments, the anti-PD-L1 antibody or antigen-binding fragment thereof comprises a light chain variable region (VL) and a heavy chain variable region (VH), wherein said VL comprises VL-CDR1, VL-CDR2 and VL-CDR3 polypeptides and VH comprises VH-CDR1, VH-CDR2 and VH-CDR3 polypeptides which are selected from the group consisting of:
(a) VL-CDR1 is TGTSSDVGGYNYVS, VL-CDR2 is DVSNRPS, VL-CDR3 is SSYTSSSTRV, VH-CDR1 is SYIMM, VH-CDR2 is SIYPSGGITFYADTVKG, and VH-CDR3 is IKLGTVTTVDY;
(b) VL-CDR1 is RASQDVSTAVA, VL-CDR2 is SASFLYS, VL-CDR3 is QQYLYHPAT, VH-CDR1 is GFTFSDSWIH, VH-CDR2 is AWISPYGGSTYYADSVKG, and VH-CDR3 is RHWPGGFDY; and
(c) VL-CDR1 is RASQRVSSSYLA, VL-CDR2 is DASSRAT, VL-CDR3 is QQYGSLPWT, VH-CDR1 is RYWMS, VH-CDR2 is NIKQDGSEKYYVDSVKG, and VH-CDR3 is EGGWFGELAFDY.

Anti-PD-L1 antibodies and antigen-binding fragments thereof can comprise polypeptides comprising the variable light chains or variable heavy chains described herein (e.g. the variable light chains within SEQ ID NOs: 24-26 or the variable heavy chains within SEQ ID NOs: 21-23). Anti-PD-1 antibodies and polypeptides can also comprise both a variable light chain (e.g., a variable light chain within SEQ ID NOs: 24-26) and a variable heavy chain (e.g., a variable heavy chain within SEQ ID NOs: 21-23).

In some embodiments, the anti-PD-L1 antibodies and antigen binding fragments thereof can comprise polypeptides comprising the variable light chain of SEQ ID NO: 24 or variable heavy chain of SEQ ID NO: 21. In some embodiments, the anti-PD-L1 antibodies and antigen binding fragments thereof can comprise polypeptides comprising the light chain of SEQ ID NO: 24 and the heavy chain of SEQ ID NO: 21.

In some embodiments, the anti-PD-L1 antibodies and antigen binding fragments thereof can comprise polypeptides comprising the heavy chain of SEQ ID NO: 21 in which the C-terminal lysine (K) is absent. In some embodiments, the anti-PD-L1 antibodies and antigen binding fragments thereof can comprise polypeptides comprising the light chain of SEQ ID NO: 24 and the heavy chain of SEQ ID NO: 21 in which the C-terminal lysine (K) is absent.

Amino acid sequences of certain anti-PD-L1 antibodies are provided in Tables 1-4 below:

TABLE 1
Variable heavy chain CDR amino acid sequences

| Antibody | VH-CDR1 | VH-CDR2 | VH-CDR3 |
|---|---|---|---|
| Avelumab | SYIMM (SEQ ID | SIYPSGGITFYADTVKG | IKLGTVTTVDY (SEQ |
| | NO: 3) | (SEQ ID NO: 4) | ID NO: 5) |
| Atezolizumab | GFTFSDSWIH | AWISPYGGSTYYADSVKG | RHWPGGFDY (SEQ ID |
| | (SEQ ID NO: 6) | (SEQ ID NO: 7) | NO: 8) |
| Durvalumab | RYWMS (SEQ ID | NIKQDGSEKYYVDSVKG | EGGWFGELAFDY (SEQ |
| | NO: 9) | (SEQ ID NO: 10) | ID NO: 11) |

TABLE 2
Variable light chain CDR amino acid sequences

| Antibody | VL-CDR1 | VL-CDR2 | VL-CDR3 |
|---|---|---|---|
| Avelumab | TGTSSDVGGYNYVS | DVSNRPS (SEQ ID | SSYTSSSTRV (SEQ ID |
| | (SEQ ID NO: 12) | NO: 13) | NO: 14) |
| Atezolizumab | RASQDVSTAVA | SASFLYS (SEQ ID | QQYLYHPAT (SEQ ID |
| | (SEQ ID NO: 15) | NO: 16) | NO: 17) |
| Durvalumab | RASQRVSSSYLA | DASSRAT (SEQ ID | QQYGSLPWT (SEQ ID |
| | (SEQ ID NO: 18) | NO: 19) | NO: 20) |

TABLE 3
Full-length amino acid sequences of heavy chains

| | Amino Acid Sequence |
|---|---|
| Avelumab | EVQLLESGGGLVQPGGSLRLSCAASGFTFSSYIMMWVRQAPGKGLEWV |
| | SSIYPSGGITFYADTVKGRFTISRDNSKNTLYLQMNSLRAEDTAVYYCAR |
| | IKLGTVTTVDYWGQGTLVTVSSASTKGPSVFPLAPSSKSTSGGTAALGC |
| | LVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYT |
| | QKSLSLSPGK (SEQ ID NO: 21) |
| Atezolizumab | EVLVESGGGLVQPGGSLRLSCAASGFTFSDSWIHWVRQAPGKGLEWV |
| | AWISPYGGSTYYADSVKGRFTISADTSKNTAYLQMNSLRAEDTAVYYC |
| | ARRHWPGGFDYWGQGTLVTVSSASTKGPSVFPLAPSSKSTSGGTAALG |
| | CLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNA |
| | KTKPREEQYASTYRVVSVLTVLHQDWLNGKEYKCKVSNKALPAPIEKTI |
| | SKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALH |
| | NHYTQKSLSLSPGK (SEQ ID NO: 22) |
| Durvalumab | EVLVESGGGLVQPGGSLRLSCAASGFTFSRYWMSWVRQAPGKGLEW |
| | VANIKQDGSEKYYVDSVKGRFTISRDNAKNSLYLQMNSLRAEDTAVYY |
| | CAREGGWFGELAFDYWGQGTLVTVSSASTKGPSVFPLAPSSKSTSGGTA |
| | ALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPEFEGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPASIEKTI |
| | SKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALH |
| | NHYTQKSLSLSPGK (SEQ ID NO: 23) |

TABLE 4
Full-length amino acid sequences of light chains

| | Full-Length Amino Acid Sequence |
|---|---|
| Avelumab | QSALTQPASVSGSPGQSITISCTGTSSDVGGYNYVSWYQQHPGKAPKLM |
| | IYDVSNRPSGVSNRFSGSKSGNTASLTISGLQAEDEADYYCSSYTSSSTR |
| | VFGTGTKVTVLGQPKANPTVTLFPPSSEELQANKATLVCLISDFYPGAVT |
| | VAWKADGSPVKAGVETTKPSKQSNNKYAASSYLSLTPEQWKSHRSYSC |
| | QVTHEGSTVEKTVAPTECS (SEQ ID NO: 24) |
| Atezolizumab | DIQMTQSPSSLSASVGDRVTITCRASQDVSTAVAWYQQKPGKAPKLLIY |
| | SASFLYSGVPSRFSGSGSGTDFTLTISSLQPEDFATYYCQQYLYHPATFG |
| | QGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 25) |
| Durvalumab | EIVLTQSPGTLSLSPGERATLSCRASQRVSSSYLAWYQQKPGQAPRLLIY |
| | DASSRATGIPDRFSGSGSGTDFTLTISRLEPEDFAVYYCQQYGSLPWTFG |
| | QGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 26) |

In some embodiments, an anti-PD-L1 antibody or antigen-binding fragment thereof comprises a variable heavy chain and a variable light chain of a full length heavy and corresponding full length light chain provided herein.

In some embodiments, an anti-PD-L1 antibody or antigen-binding fragment thereof comprises the CDR sequences of avelumab (i.e. SEQ ID NOs: 3, 4, 5, 12, 13, and 14), atezolizumnab (i.e. SEQ ID NOs: 6, 7, 8, 15, 16, and 17), and durvalumab (i.e. SEQ ID NOs: 9, 10, 11, 18, 19, and 20) and blocks the interaction between PD-1 and PD-L1. In some embodiments, an anti-PD-L1 antibody or antigen-binding fragment thereof comprises the CDR sequences of avelumab (i.e. SEQ ID NOs: 3, 4, 5, 12, 13, and 14), atezolizumab (i.e. SEQ ID NOs: 6, 7, 8, 15, 16, and 17), and durvalumab (i.e. SEQ ID NOs: 9, 10, 11, 18, 19, and 20) and blocks the interaction between PD-L1 and PD-L2. In some embodiments, an anti-PD-L1 antibody or antigen-binding fragment thereof comprises the CDR sequences of avelumab (i.e. SEQ ID NOs: 3, 4, 5, 12, 13, and 14), atezolizumab (i.e. SEQ ID NOs: 6, 7, 8, 15, 16, and 17), and durvalumab (i.e. SEQ ID NOs: 9, 10, 11, 18, 19, and 20) and releases PD-1 pathway-mediated inhibition of an immune response, e.g., an anti-tumor immune response.

In some embodiments, an anti-PD-L1 antibody, or antigen-binding fragment thereof, comprises the heavy and light chain sequences of avelumab (i.e., SEQ ID NOs: 21 and 24), atezolizumab (i.e. SEQ ID NOs: 22 and 25), or durvalumab (i.e. SEQ ID NOs: 23 and 26).

Avelumab, its sequence, and many of its properties have been described in WO 2013/079174, where it is designated A09-246-2 having the heavy and light chain sequences according to SEQ ID NOs: 32 and 33. It is frequently observed, however, that in the course of antibody production the C-terminal lysine (K) of the heavy chain is cleaved off. This modification has no influence on the antibody-antigen binding. Therefore, in some embodiments, an anti-PD-L1 antibody, or antigen-binding fragment thereof, comprises the heavy chain sequence of SEQ ID NO: 21 in which the C-terminal lysine (K) is absent and the light chain sequence of SEQ ID NO: 24; the heavy chain sequence of SEQ ID NO: 22 in which the C-terminal lysine (K) is absent and the light chain of SEQ ID NO: 25; or the heavy chain of SEQ ID NO: 23 in which the C-terminal lysine (K) is absent and the light chain of SEQ ID NO: 26.

### ITEMS OF THE INVENTION

The present invention also comprises the following items:
1. A method for treating a human patient having an advanced solid malignancy comprising administering to the patient, in need thereof, a therapeutically effective amount of Debio 1143 and a therapeutically effective amount of an anti-PD-L1 antibody, or antigen-binding fragment thereof.
2. The method of item 1, wherein the anti-PD-L1 antibody is an anti-PD-L1 IgG1 antibody.
3. The method of item 2, wherein the anti-PD-L1 IgG1 antibody is avelumab.
4. The method of any one of items 1-3, wherein the therapeutically effective amount of Debio 1143 is about 75 to about 250 mg per day.
5. The method of any one of items 1-3, wherein the therapeutically effective amount of Debio 1143 is about 75 mg per day.
6. The method of any one of items 1-3, wherein the therapeutically effective amount of Debio 1143 is about 100 mg per day.
7. The method of any one of items 1-3, wherein the therapeutically effective amount of Debio 1143 is about 150 mg per day.
8. The method of any one of items 1-3, wherein the therapeutically effective amount of Debio 1143 is about 200 mg per day.
9. The method of any one of items 1-3, wherein the therapeutically effective amount of Debio 1143 is about 250 mg per day.
10. The method of any one of items 1-9, wherein the Debio 1143 is administered orally.
11. The method of any one of items 1-10, wherein the Debio 1143 is administered in capsular form.
12. The method of any one of items 1-11, wherein the Debio 1143 is administered orally as a capsule containing 75 mg Debio 1143.
13. The method of any one of items 1-11, wherein the Debio 1143 is administered orally as a capsule containing 100 mg Debio 1143.
14. The method of any one of items 1-13, wherein the therapeutically effective amount of Debio 1143 is administered as one dose one time per day.
15. The method of any one of items 1-13, wherein the therapeutically effective amount of Debio 1143 is divided into multiple doses that are administered as multiple doses two, three, or four times per day.
16. The method of any one of items 1-15, wherein the Debio 1143 is administered once daily for 10 consecutive days.
17. The method of any one of items 1-15, wherein the method of treatment comprises a 28 day cycle comprising administering the Debio 1143 for 10 consecutive days, followed by administering no Debio 1143 for 4 consecutive days.
18. The method of any one of items 1-17, wherein the anti-PD-L1 antibody is avelumab and the therapeutically effective amount of avelumab is about 10 mg/kg.
19. The method of item 18, wherein the avelumab is administered once every two weeks.
20. The method of any one of items 18 and 19, wherein the avelumab is administered on days 1 and 15 of a 28-day cycle.
21. The method of any one of items 1-20, wherein the anti-PD-L1 antibody is administered intravenously.
22. The method of any one of items 18-21 further comprising administering an antihistamine (anti-H₁) and acetaminophen to the patient prior to administering the avelumab.
23. The method of item 22, wherein the antihistamine (anti-H1) and acetaminophen are administered to the patient about 30 minutes to about 60 minutes prior administering the avelumab.
24. The method of any one of items 22 and 23, wherein the antihistamine (anti-H1) and acetaminophen are administered prior to each of the first four administrations of avelumab.
25. The method of any one of items 22-24, wherein the antihistamine (anti-H1) is diphenhydramine.
26. The method of item 25, wherein about 25 to about 50 mg diphenhydramine is administered.
27. The method of any one of items 1-26, wherein the advanced solid malignancy is one or more selected from the group consisting of Lung Cancer, Head and Neck cancer, bladder cancer, kidney cancer, skin melanoma, colorectal cancer, ovarian cancer, breast cancer, non-Hodgkin and/or Hodgkin lymphomas.
28. The method of any one of items 1-27, wherein the advanced solid malignancy is Non-Small Cell Lung Cancer.
29. The method of any one of items 1-28, wherein the advanced solid malignancy is advanced or metastatic Non-Small Cell Lung Cancer.
30. The method of any one of items 1-29, wherein the patient previously received platinum-based therapy for treatment of the advanced solid malignancy.
31. The method of items 1-30, wherein the patient has stage IIIB or stage IV Non-Small Cell Lung Cancer.
32. A method of treating a human patient having advanced or metastatic Non-Small Cell Lung Cancer comprising administering to the patient, in need thereof, about 75 mg to about 250 mg of Debio 1143 and about 10 mg/kg avelumab.
33. The method of item 32, wherein the patient with advanced or metastatic Non-Small Cell Lung Cancer previously received platinum-based therapy for the Non-Small Cell Lung Cancer.
34. The method of item 33, wherein the patient is orally administered the Debio 1143.
35. The method of item 34, wherein the Debio 1143 is provided in capsular form.
36. The method of item 34, wherein the patient is orally administered the Debio 1143 for 10 consecutive days.
37. The method of item 34, wherein the method of treatment comprises a 28 day cycle comprising
   (a) administering the Debio 1143 for 10 consecutive days; and
   (b) administering no Debio 1143 for 4 consecutive days.
38. The method of item 32, wherein the avelumab is administered once every two weeks.
39. The method of item 32, wherein the avelumab is administered on days 1 and 15 of a 28-day cycle.
40. The method of item 37, wherein the avelumab is administered on days 1 and 15 of the 28-day cycle.
41. The method of item 38, wherein the avelumab is administered intravenously.
42. The method of item 39, wherein the avelumab is administered intravenously.
43. The method of item 40, wherein the avelumab is administered intravenously.
44. The method of item 38 further comprising administering an antihistamine (anti-H1) and acetaminophen to the patient prior to administering the avelumab.
45. The method of item 39 further comprising administering an antihistamine (anti-H1) and acetaminophen to the patient prior to administering the avelumab.
46. The method of item 40 further comprising administering an antihistamine (anti-H1) and acetaminophen to the patient prior to administering the avelumab.
47. The method of item 44, wherein the antihistamine (anti-H1) and acetaminophen are administered to the patient about 30 minutes to about 60 minutes prior to administering the avelumab.
48. The method of item 45, wherein the antihistamine (anti-H1) and acetaminophen are administered to the patient about 30 minutes to about 60 minutes prior to administering the avelumab.
49. The method of item 46, wherein the antihistamine (anti-H1) and acetaminophen are administered to the patient about 30 minutes to about 60 minutes prior to administering the avelumab.
50. The method of item 47, wherein the antihistamine (anti-H1) and acetaminophen are administered prior to each of the first four administrations of avelumab.
51. The method of item 48, wherein the antihistamine (anti-H1) and acetaminophen are administered prior to each of the first four administrations of avelumab.
52. The method of item 49, wherein the antihistamine (anti-H1) and acetaminophen are administered prior to each of the first four administrations of avelumab.
53. The method of item 50, wherein the antihistamine (anti-H1) is diphenhydramine.
54. The method of item 51, wherein the antihistamine (anti-H1) is diphenhydramine.
55. The method of item 52, wherein the antihistamine (anti-H1) is diphenhydramine.
56. The method of item 53, wherein about 25 to about 50 mg diphenhydramine is administered.
57. The method of item 54, wherein about 25 to about 50 mg diphenhydramine is administered.
58. The method of item 55, wherein about 25 to about 50 mg diphenhydramine is administered.
59. The method of item 34, wherein the method of treatment comprises a 28 day cycle comprising
   (a) administering the Debio 1143 for a first 10 consecutive day period;
   (b) administering no Debio 1143 for a first 4 consecutive day period;
   (c) administering the Debio 1143 for a second 10 consecutive day period; and
   (d) administering no Debio 1143 for a second 4 consecutive day period.
60. The method of item 59, wherein the avelumab is administered once every two weeks.
61. The method of item 59, wherein the avelumab is administered on days 1 and 15 of the 28-day cycle.
62. The method of item 60, wherein the avelumab is administered intravenously.
63. The method of item 61, wherein the avelumab is administered intravenously.
64. The method of item 62 further comprising administering an antihistamine (anti-H1) and acetaminophen to the patient prior to administering the avelumab.
65. The method of item 63 further comprising administering an antihistamine (anti-H1) and acetaminophen to the patient prior to administering the avelumab.
66. The method of item 64, wherein the antihistamine (anti-H1) and acetaminophen are administered to the patient about 30 minutes to about 60 minutes prior to administering the avelumab.
67. The method of item 65, wherein the antihistamine (anti-H1) and acetaminophen are administered to the patient about 30 minutes to about 60 minutes prior to administering the avelumab.
68. The method of item 66, wherein the antihistamine (anti-H1) and acetaminophen are administered prior to each of the first four administrations of avelumab.
69. The method of item 67, wherein the antihistamine (anti-H1) and acetaminophen are administered prior to each of the first four administrations of avelumab.
70. The method of item 68, wherein the antihistamine (anti-H1) is diphenhydramine.
71. The method of item 69, wherein the antihistamine (anti-H1) is diphenhydramine.
72. The method of item 70, wherein about 25 to about 50 mg diphenhydramine is administered.
73. The method of item 71, wherein about 25 to about 50 mg diphenhydramine is administered
74. A method of treating a human patient having advanced or metastatic Non-Small Cell Lung Cancer after platinum-based therapy comprising orally administering to the patient, in need thereof, about 75 mg to about 250 mg of Debio 1143 and intravenously about 10 mg/kg avelumab, wherein the method of treatment comprises a 28 day cycle comprising
   (a) administering the Debio 1143 for a first 10 consecutive day period;
   (b) administering the no Debio 1143 for a first 4 consecutive day period;
   (c) administering the Debio 1143 for a second 10 consecutive day period;
   (d) administering no Debio 1143 for a second 4 consecutive day period;
   (e) administering the avelumab on day 1 of the 28-day cycle; and
   (f) administering the avelumab on day 15 of the 28-day cycle.
75. The method of item 74, wherein the Debio 1143 is administered in capsular form.
76. The method of item 74 further comprising administering an antihistamine (anti-H1) to the patient prior to administering the avelumab.
77. The method of item 74 further comprising administering acetaminophen to the patient prior to administering the avelumab.
78. The method of item 74 further comprising administering an antihistamine (anti-H1) and acetaminophen to the patient prior to administering the avelumab.
79. The method of item 76, wherein the antihistamine (anti-H1) is administered to the patient about 30 minutes to about 60 minutes prior to administering the avelumab.
80. The method of item 77, wherein the acetaminophen is administered to the patient about 30 minutes to about 60 minutes prior to administering the avelumab
81. The method of item 78, wherein the antihistamine (anti-H1) and acetaminophen are administered to the patient about 30 minutes to about 60 minutes prior to administering the avelumab.
82. The method of item 79, wherein the antihistamine (anti-H1) is diphenhydramine
83. The method of item 81, wherein the antihistamine (anti-H1) is diphenhydramine.
84. The method of item 82, wherein about 25 to about 50 mg diphenhydramine is administered
85. The method of item 83, wherein about 25 to about 50 mg diphenhydramine is administered.
86. The method of any one of items 32-85, wherein the therapeutically effective amount of Debio 1143 is administered as one dose one time per day.
87. The method of any one of items 32-85, wherein the therapeutically effective amount of Debio 1143 is divided into multiple doses that are administered as multiple doses two, three, or four times per day.
88. The method of any one of items 32-74, wherein the patient previously received platinum-based therapy for treatment of the Non-Small Cell Lung Cancer.
89. The method of any one of items 30 and 74-88, wherein the platinum-based therapy comprised administering one of more platinum-based agents selected from the group consisting of cisplatin, carboplatin, and oxaliplatin.
90. The method of any one of items 30 and 89, wherein the patient has relapsed or progressed after being administered the platinum-based therapy but before being administered the Debio 1143.

### EXAMPLES

It is understood that the examples and embodiments described herein are for illustrative purposes only and that various modifications or changes in light thereof will be suggested to persons skilled in the art and are to be included within the spirit and purview of this application.

### Example 1: Debio 1143-Induced T Cell Activation

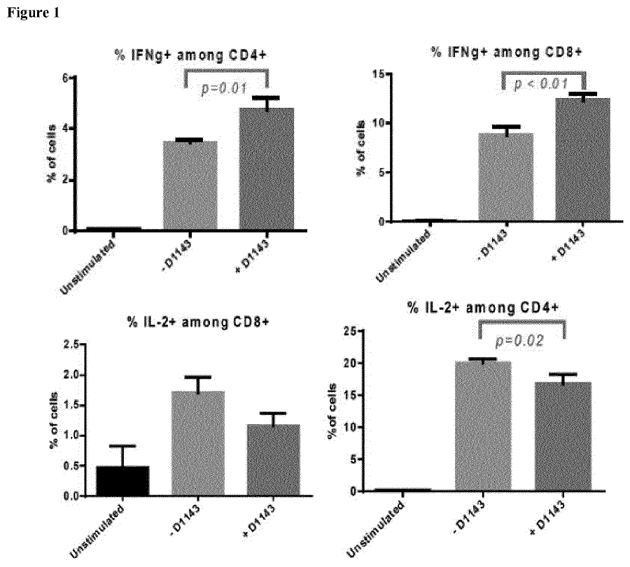

To test the effect of Debio 1143 on activation of human T cells, PBMCs freshly isolated from healthy human donors were stimulated ex vivo with anti-CD3/CD28 antibodies for 24 in the presence of 10 μM Debio 1143, or control treatments without stimulation and Debio 1143 incubation. In brief, cytometric analyses showed that CD3/CD28 stimulation increased the percentage of IFNγ+CD4+ and IFNγ+CD8+ T-cells at 24 hours, and that addition of Debio 1143 treatment further added a significant increase in T cell activation (FIG. 1).

### Example 2: Combination of Debio 1143 with an Anti-CTLA4 Antibody and IDO Inhibitor

The therapeutic efficacy of Debio 1143 combined with an anti-CTLA4 antibody was tested in a TS/A breast cancer mouse syngeneic model. Further, the therapeutic efficacy of Debio 1143 combined with an IDO inhibitor (INCB024360) was tested in a CT-26 colorectal cancer mouse syngeneic model. In both of these cases, the combination therapy did not lead to a statistically significant improvement compared to the respective monotherapies.

Thus, providing a combination therapy comprising Debio 1143 which results in an additive or synergistic effect may require the non-obvious selection of specific immune checkpoint regulator targets. Simply combining Debio 1143 with any immunotherapy is not sufficient to obtain an improved efficacy or an effective combination therapy which can be used to treat any cancer.

### Example 3: Dose-Dependency of Debio 1143 in a Combination Therapy

To test whether Debio 1143 dose-dependently enhanced anti-PD1 anti-tumor activity, the tolerability of oral Debio 1143 at 200 and 300 mg/kg for 5 days/week in combination with 10 mg/kg anti-PD1 twice weekly was tested for a total duration of 2 weeks in tumor-free female C57BL/6 mice. In contrast to the 300 mg/kg dose, combination treatment with the 200 mg/kg dose was well tolerated with only minor body weight loss on treatment and good recovery of all mice upon dosing completion. Thus, 200 mg/kg was chosen as the highest dose level for the efficacy study evaluating the combination in mice bearing s.c. B16F10 melanoma tumors.

C57BL/6 mice were s.c. injected with 2×10⁵ B16F10 tumor cells (see, for example, Bobek et al., 2010. Anticancer Res. 30(12):4799-803) and treatments started at a mean group tumor size of 94 mm³ (n=8/group). Debio 1143 at 100 and 200 mg/kg p.o. was given on 5 days/week in combination with 10 mg/kg i.p. anti-PD1 twice weekly and compared to treatment with vehicle plus isotype antibody. In addition, the 100 mg/kg dose given twice weekly in combination with anti-PD1 at 10 mg/kg i.p. twice weekly was tested.

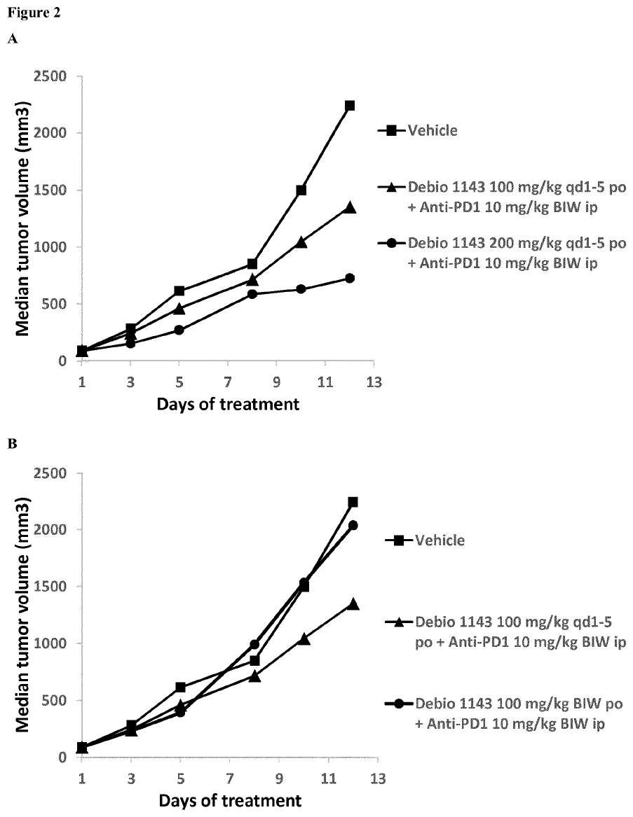

By day 19 from tumor inoculation, treatment with 100 mg/kg Debio 1143 QD1-5 combined with anti-PD-1 at 10 mg/kg also displayed no significant antitumor activity with mean tumor volume of 1049 mm³ (TGI=31%, P=0.155), as compared to the vehicle group (mean tumor volume 1529 mm³; FIG. 2A). However, Debio 1143 at 200 mg/kg in combination with 10 mg/kg anti-PD-1 produced significant antitumor activity (594 mm³; TGI=61%; P=0.005; FIG. 2A). Bi-weekly Debio 1143 at 100 mg/kg (mean tumor volume: 1396 mm³, TGI=9%, P=0.757) in combination with anti-PD-1 at 10 mg/kg also did not produce any antitumor activity as compared to the vehicle group (FIG. 2B). The administration of Debio 1143 at 100 mg/kg over 5 days/week was considered superior to bi-weekly administration for the combination with anti-PD-1 at 10 mg/kg.

In view of the above, the subsequent mouse studies testing the efficacy of Debio 1143 with an anti-PD-L1 antibody were performed by orally administering Debio 1143 5 days/week and intraperitoneally administering the anti-PD-L1 antibody twice a week.

### Example 4: Combination of Debio 1143 with an Anti-PD-L1 Antibody

The anti-tumor activity of an anti-PD-L1 antibody (5 mg/kg BIW i.p.; clone 10F.9G2, BioXcell) was tested either alone or in combination with Debio 1143 (100 mg/kg QD1-5 p.o.) in MBT-2 immunocompetent syngeneic mouse model of bladder cancer in C3H/HeNCrl mice over 3 weeks (see, for example, Shimazui et al., 2013. Int J Oncol. 42(2):543-8). Each mouse was inoculated subcutaneously at the right flank region with MBT-2 tumor cells (2×10⁵) in 0.1 ml of PBS for tumor development. The treatments were started when the mean tumor size reached 101 mm³ (n=8/group).

While bi-weekly anti-PD-L1 at 5 mg/kg i.p. displayed no anti-tumor activity, and Debio 1143 at 100 mg/kg p.o. QD1-5 only weak anti-tumor activity, the combination demonstrated pronounced anti-tumor activity (FIG. 3) and significantly prolonged survival of the mice. Mean body weight increased in all groups, indicating that treatments were well-tolerated at this dose level and schedule.

The combination of Debio 1143 and anti-PD-L1 antibody significantly decreased tumor growth in comparison to the vehicle control and anti-PD-L1 alone (p<0.001 according to the two-sided t-test with equal variance) as well as Debio 1143 alone (p<0.01 according to the two-sided t-test with equal variance).

Based on the single agent activities, the Bliss independence model predicted 53% tumor growth inhibition (TGI) for additive effects of the combination on day 13 of treatment. However, the combination of Debio 1143 and anti-PD-L1 resulted in 80% TGI at that time point, with a combination index of 0.66, indicative of synergy (Foucquier & Guedj, 2015. Pharmacol Res Perspect. 3(3):c00149).

### Example 5: Debio 1143 Dosing Trial in Human Patients with Advanced Solid Tumors

The trial has enrolled patients with advanced solid tumors (up to 24 evaluable patients planned). The patients have advanced solid malignancies and are not eligible for standard therapy or standard therapy has failed them.

Debio 1143 is being administered once daily for 10 consecutive days every 2 weeks (i.e. 10 days on, 4 days off) at a starting dose of 100 mg. Dose increments for subsequent dose groups is 50 mg (i.e. 100, 150, 200, 250 mg). Patients should fast 2 hours before dosing and should fast at least 1 hour post dose. Water is permitted freely. Further dose levels may be considered if pharmacokinetic (“PK”) analysis identifies lower drug exposure of either Debio 1143 or avelumab.

Avelumab (an anti-PD-L1 antibody) is administered at 10 mg/kg through an i.v. infusion over one hour, Q2W (i.e. on days 1 and 15 of a 28-day cycle). The avelumab dose will not be escalated unless PK analysis reveals a significant interaction lowering exposure to avelumab. Lower doses of avelumab will not be explored; dose reduction will not be allowed.

Based on the review of toxicity and PK data at the first dose levels, feasibility and appropriateness of the dosing schedule (10 days on/4 days off) along with the standard dose of avelumab will be assessed. If deemed required by the study Safety Monitoring Committee or if two patients experience Dose Limiting Toxicities already at the initial dose level (100 mg), an alternative dosing schedule (e.g. 5 days on/2 days off every week) will be considered.

The dose of avelumab is calculated based on the weight of the patient determined within 72 hours prior to administration. As compared to the previous administration, the same dose may be used as long as weight change is ≤10% from the previous administration.

Prior to infusion, avelumab is diluted with 0.9% (or 0.45%, if needed) saline solution supplied as infusion bag. Dosage is 10 mg/kg body weight administered i.v. over 1 hour (−10/+20 minutes, i.e. 50 to 80 minutes) once every 2 weeks, on days 1 and 15 of each cycle. A manual of preparation describes in detail infusion bags and medical devices to be used for the preparation of the dilution and subsequent administration.

Premedication with an antihistamine (anti-H1) and with acetaminophen (paracetamol) approximately 30 to 60 minutes prior to each dose of avelumab is mandatory for the first 4 infusions (e.g. 25-50 mg diphenhydramine and 500-650 mg acetaminophen (paracetamol) i.v. or oral equivalent). This regimen may be modified based on local treatment standards and guidelines, as appropriate. Premedication should be administered for subsequent avelumab doses based upon clinical judgment and presence/severity of prior infusion reactions.

The primary endpoint of this study is the maximum tolerated dose with an estimated probability of dose limiting toxicity (“DLT”) below 30%. Based on this dose, the recommended Phase-II dose will be determined also taking into account overall cumulative safety/tolerability, PK, and efficacy data. DLT is defined as any of the following Adverse Events (AEs) during the first treatment cycle (i.e. 4 weeks or longer in case of dosing delays) if deemed related to treatment:
grade 3 or 4 febrile neutropenia or any grade 4 neutropenia of >5 days duration
grade 4 thrombocytopenia (<25000/mm3) or grade 3 (<50000/mm3), if associated with medically concerning bleeding
any grade ≥3 non-hematologic laboratory value if:
   medical intervention is required to treat the patient or
   the abnormality leads to hospitalization or persists for >7 days or
   deemed clinically significant by the treating investigator
any non-hematologic toxicity (not laboratory) of grade 3 or 4, except:
   grade 3 infusion-related reaction resolving within 6 h from the end of infusion
   transient (≤6 hours) grade 3 flu-like symptoms or fever, which is controlled with medical management
   transient (≤24 hours) grade 3 fatigue, local reactions, headache, nausea, emesis that resolve to grade ≤1
   grade 3 diarrhea or grade 3 skin toxicity that resolves to grade ≤1 in ≤7 days after medical management (e.g. immunosuppressant treatment) has been initiated
   grade 3 autoimmune thyroid-related toxicity that abates to grade 52 within 7 days of initiating therapy
   local pain, irritation, or rash localized at sites of known or suspected tumor (tumor flare phenomenon)
any grade ≥2 uveitis or eye pain that does neither respond to topical therapy nor abate to grade 1 within the avelumab re-treatment period OR that required systemic treatment
any grade ≥2 pneumonitis or interstitial lung disease that did not resolve with dose delay and systemic steroids.
any toxicity related to Debio 1143 or avelumab that requires a dosing delay of >2 weeks, dose reduction, or premature discontinuation of any of the two
any other drug related AE that in the opinion of the investigator is of potential clinical significance so that further dose-escalation would expose patients to unacceptable risk.

The secondary endpoints of this study are:
Incidence and severity of treatment-emergent AEs and laboratory abnormalities, graded according to NCI-CTCAE version 4.03 criteria;
Incidence of premature treatment discontinuations and treatment modifications due to AEs and laboratory abnormalities;
Best change in tumor size;
Tumor response determined according to RECIST version 1.1 criteria:
   ORR at end of cycle 6
   Best overall response (BOR)
   Duration of response
   Disease control rate
   PFS median and rate at 6 months, 1 year, and 2 years from treatment initiation
OS median and rate at 6 months, 1 year, and 2 years from treatment initiation
PK parameters. In addition, post-hoc estimate of the area under the curve (AUC) of Debio 1143 and avelumab as well as target occupancy (TO) of avelumab in combination with Debio 1143 will be evaluated.

### Example 6: Debio 1143 Dosing Trial in Human Patients with Non-Small Cell Lung Cancer

Patients with advanced or metastatic Non-Small Cell Lung Cancer (NSCLC) whose cancer has progressed after one line of platinum-based chemotherapy have been enrolled. The patients have histologically or cytologically confirmed NSCLC of stage 111B or IV (per 7th International Association for the Study of Lung Cancer classification) that has progressed after one line of platinum-containing doublet chemotherapy (i.e. adjuvant treatment with a platinum-containing regimen is not sufficient for eligibility because not received in the context of a metastatic disease).

Once the Recommended Phase-II dose and schedule are established, Non-Small Cell Lung Cancer patients will be enrolled in an expansion cohort and treated at this dose level along with avelumab unless disease progression or severe toxicity is observed. In individual cases of severe toxicity, the Debio 1143 dose may be reduced by decrements of 50 mg (except the 100 mg dose for which the decrement would be 25 mg).

The primary endpoint of this study is the Objective Response Rate (“ORR”) as per RECIST version 1.1. The secondary endpoints of this study are the same as those listed above in Example 4.

### Example 7: Results of Treatment on Patient A

A 64 years-old woman treated in a North American Hospital was diagnosed on 10 Nov. 2016 with a stage IV (metastatic) adenocarcinoma of the lungs (non-squamous NSCLC).

Relevant medical history included, ongoing Diabetes mellitus type II and hypertension and history of right-leg DVT and associated Pulmonary embolism.

She started on 24 Nov. 2016 first-line treatment with standard Chemotherapy (PemCis regimen) for four cycles and achieved a best overall response of stable disease (SD) being switched to Pemetrexed maintenance on 13 Mar. 2017 due to PemCis toxicity. Progressive disease (PD) occurred on 13 Dec. 2017 despite this treatment.

On 17 Jan. 2018 she consented to participate in the Debio1143-105 study, at baseline she presented right sided thoracic pain (under opioids treatment), dyspnoea, anorexia, anxiety and fatigue. She presented an Eastern Cooperative Oncology Group Performance Status (ECOG-PS)=1 (mildly symptomatic).

On 8 Feb. 2018, she started treatment with Debio 1143 at 150 mg/daily orally from Days 1 to 10 and 15 to 24 together with avelumab infusions at 10 mg/kg on Days 1 and 15 every four weeks (q4w). Her baseline CT scan, done before treatment started on 24 Jan. 2018, showed multiple pulmonary and brain metastases as non-measurable disease and two target measurable lesions: a left pulmonary nodule measuring 42 mm and an hiliar mass of 41 mm, for a total of 83 mm of measurable disease at baseline.

She received two cycles of study treatment with good tolerance and no dose-limiting toxicity (DLT) and good treatment compliance. CT scan evaluation before Cycle 3 was done on 29 Mar. 2018, showed mild decrease in lesions size to 37 mm each, reaching criteria for stable disease as per RECIST v1.1. She continued treatment for additionally two cycles and a new CT scan was performed on 29 May 2018 showing further tumor shrinkage to 28 and 23 μm, respectively for a total of 51 mm. Therefore a 38.5% decrease in target lesions as compared to baseline measurements, thus Partial response was achieved (PR). This response was further confirmed in the last available disease assessment done on 19 Jul. 2018. The patient is now ECOG-PS=0 (asymptomatic) and is still ongoing treatment.

Thus, the combination therapy of the present invention is effective at treating cancer (in particular, NSCLC).

### Example 8: Safety of the Debio 1143 Dosages Tested in the Ongoing Clinical Trial

A daily dose of 100-250 mg Debio 1143 for 10 consecutive days every 2 weeks in combination with Avelumab administered at 10 mg/kg through an i.v. infusion over one hour, Q2W (i.e. on days 1 and 15 of a 28-day cycle) has been well-tolerated by the patients of the ongoing clinical trial.

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A combination product comprising:
(i) Debio 1143; and
(ii) an anti-PD-L1 antibody or antigen-binding fragment thereof.

2. The combination product according to claim 1, wherein the combination product is a pharmaceutical combination product and further comprises a pharmaceutically acceptable carrier, diluent, excipient and/or adjuvant.

3. A pharmaceutical composition comprising:
(i) Debio 1143;
(ii) an anti-PD-L1 antibody or antigen-binding fragment thereof; and
(iii) a pharmaceutically acceptable carrier, diluent, excipient and/or adjuvant.

4. The combination product according to any one of claims 1-2, or the pharmaceutical composition according to claim 3, wherein the antibody or antigen-binding fragment thereof mediates antibody-dependent cellular cytotoxicity.

5. The combination product according any one of claims 1, 2 or 4, or the pharmaceutical composition according to any one of claims 3-4, wherein the antibody or antigen-binding fragment thereof comprises a light chain variable region (VL) and a heavy chain variable region (VH), wherein said VL comprises VL-CDR1, VL-CDR2 and VL-CDR3 polypeptides and VH comprises VH-CDR1, VH-CDR2 and VH-CDR3 polypeptides which are selected from the group consisting of:
(a) VL-CDR1 is TGTSSDVGGYNYVS, VL-CDR2 is DVSNRPS, VL-CDR3 is SSYTSSSTRV, VH-CDR1 is SYIMM, VH-CDR2 is SIYPSGGITFYADTVKG, and VH-CDR3 is IKLGTVTTVDY;
(b) VL-CDR1 is RASQDVSTAVA, VL-CDR2 is SASFLYS, VL-CDR3 is QQYLYHPAT, VH-CDR1 is GFTFSDSWIH, VH-CDR2 is AWISPYGGSTYYADSVKG, and VH-CDR3 is RHWPGGFDY; and
(c) VL-CDR1 is RASQRVSSSYLA, VL-CDR2 is DASSRAT, VL-CDR3 is QQYGSLPWT, VH-CDR1 is RYWMS, VH-CDR2 is NIKQDGSEKYYVDSVKG, and VH-CDR3 is EGGWFGELAFDY;
preferably, VL-CDR1 is TGTSSDVGGYNYVS, VL-CDR2 is DVSNRPS, VL-CDR3 is SSYTSSSTRV, VH-CDR1 is SYIMM, VH-CDR2 is SIYPSGGITFYADTVKG, and VH-CDR3 is IKLGTVTTVDY.

6. The combination product according to any one of claims 1, 2, 4 or 5, or the pharmaceutical composition according to any one of claims 3-5, wherein the antibody is avelumab.

7. The combination product according to any one of claims 1-2 or 4-6, or the pharmaceutical composition according to any one of claims 3-6, wherein the anti-PD-L1 antibody and the Debio 1143 are provided in a single or separate unit dosage forms.

8. The combination product according to any one of claims 1-2 or 4-7, or the pharmaceutical composition according to any one of claims 3-7, for use as a medicament.

9. The combination product according to any one of claims 1-2 or 4-7, or the pharmaceutical composition according to any one of claims 3-7, for use in a method of treating cancer;
wherein, optionally, the cancer is selected from the group consisting of Lung Cancer, Head and Neck cancer, bladder cancer, kidney cancer, skin melanoma, colorectal cancer, ovarian cancer, breast cancer, non-Hodgkin and/or Hodgkin lymphomas, preferably Non-Small Cell Lung Cancer or bladder cancer.

10. The combination product or pharmaceutical composition for use according to claim 9, wherein the cancer is Non-Small Cell Lung Cancer, preferably stage IIIB or stage IV Non-Small Cell Lung Cancer.

11. The combination product or pharmaceutical composition for use according to any one of claims 9-10, wherein the method comprises administering about 75 to about 250 mg Debio 1143 and about 10 mg/kg of the antibody or antigen-binding fragment thereof.

12. The combination product or pharmaceutical composition for use according to any one of claims 9-11, wherein the method of treatment comprises a 28-day cycle comprising
(a) administering the Debio 1143 for a first 10 consecutive day period;
(b) administering no Debio 1143 for a first 4 consecutive day period;
(c) administering the Debio 1143 for a second 10 consecutive day period;
(d) administering no Debio 1143 for a second 4 consecutive day period;
(e) administering the avelumab on day 1 of the 28-day cycle; and
(f) administering the avelumab on day 15 of the 28-day cycle.

13. The combination product or pharmaceutical composition for use according to any one of claims 9-12, wherein a patient who is administered the combination product or pharmaceutical composition underwent at least one round of prior cancer therapy;
wherein, optionally, the cancer was resistant or became resistant to prior therapy.

14. The combination product or pharmaceutical composition for use according to any one of claims 9-13, wherein the patient who is administered the combination product or pharmaceutical composition has previously received platinum-based therapy, preferably the patient has relapsed or progressed after receiving the platinum-based therapy.

15. A kit comprising an anti-PD-L1 antibody and Debio 1143, and a package insert comprising instructions for using the anti-PD-L1 antibody and Debio 1143 to treat or delay progression of a cancer in a patient; wherein, optionally,
the kit comprises a first container, a second container and a package insert, wherein the first container comprises at least one dose of a medicament comprising the anti-PD-L1 antibody, the second container comprises at least one dose of a medicament comprising the Debio 1143, and the package insert comprises instructions for treating a subject for cancer using the medicaments; wherein, further optionally,
the instructions state that the medicaments are intended for use in treating a subject having a cancer that tests positive for PD-L1 expression, preferably by means of an immunohistochemical assay.

16. A composition comprising an anti-PD-L1 antibody for use in a method of treating cancer, wherein the composition is administered in combination with Debio 1143.

17. A composition comprising Debio 1143 for use in a method of treating cancer, wherein the composition is administered in combination with an anti-PD-L1 antibody.


SEQUENCE LISTING (26 sequences)

SEQ ID NO: 1 - protein, 288 aa, synthetic construct
  REGION 1..288: note = PD-L1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 20 aa, synthetic construct
  REGION 1..20: note = PD-L1 signal sequence
MQIPQAPWPVVWAVLQLGWR

SEQ ID NO: 3 - protein, 5 aa, synthetic construct
  REGION 1..5: note = Avelumab VH-CDR1
SYIMM

SEQ ID NO: 4 - protein, 17 aa, synthetic construct
  REGION 1..17: note = Avelumab VH-CDR2
SIYPSGGITFYADTVKG

SEQ ID NO: 5 - protein, 11 aa, synthetic construct
  REGION 1..11: note = Avelumab VH-CDR3
IKLGTVTTVDY

SEQ ID NO: 6 - protein, 10 aa, synthetic construct
  REGION 1..10: note = Atezolizumab VH-CDR1
GFTFSDSWIH

SEQ ID NO: 7 - protein, 18 aa, synthetic construct
  REGION 1..18: note = Atezolizumab VH-CDR2
AWISPYGGSTYYADSVKG

SEQ ID NO: 8 - protein, 9 aa, synthetic construct
  REGION 1..9: note = Atezolizumab VH-CDR3
RHWPGGFDY

SEQ ID NO: 9 - protein, 5 aa, synthetic construct
  REGION 1..5: note = Durvalumab VH-CDR1
RYWMS

SEQ ID NO: 10 - protein, 17 aa, synthetic construct
  REGION 1..17: note = Durvalumab VH-CDR2
NIKQDGSEKYYVDSVKG

SEQ ID NO: 11 - protein, 12 aa, synthetic construct
  REGION 1..12: note = Durvalumab VH-CDR3
EGGWFGELAFDY

SEQ ID NO: 12 - protein, 14 aa, synthetic construct
  REGION 1..14: note = Avelumab VL-CDR1
TGTSSDVGGYNYVS

SEQ ID NO: 13 - protein, 7 aa, synthetic construct
  REGION 1..7: note = Avelumab VL-CDR2
DVSNRPS

SEQ ID NO: 14 - protein, 10 aa, synthetic construct
  REGION 1..10: note = Avelumab VL-CDR3
SSYTSSSTRV

SEQ ID NO: 15 - protein, 11 aa, synthetic construct
  REGION 1..11: note = Atezolizumab VL-CDR1
RASQDVSTAVA

SEQ ID NO: 16 - protein, 7 aa, synthetic construct
  REGION 1..7: note = Atezolizumab VL-CDR2
SASFLYS

SEQ ID NO: 17 - protein, 9 aa, synthetic construct
  REGION 1..9: note = Atezolizumab VL-CDR3
QQYLYHPAT

SEQ ID NO: 18 - protein, 12 aa, synthetic construct
  REGION 1..12: note = Durvalumab VL-CDR1
RASQRVSSSYLA

SEQ ID NO: 19 - protein, 7 aa, synthetic construct
  REGION 1..7: note = Durvalumab VL-CDR2
DASSRAT

SEQ ID NO: 20 - protein, 9 aa, synthetic construct
  REGION 1..9: note = Durvalumab VL-CDR3
QQYGSLPWT

SEQ ID NO: 21 - protein, 450 aa, synthetic construct
  REGION 1..450: note = Avelumab heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 448 aa, synthetic construct
  REGION 1..448: note = Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 451 aa, synthetic construct
  REGION 1..451: note = Durvalumab heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, synthetic construct
  REGION 1..216: note = Avelumab light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - protein, 214 aa, synthetic construct
  REGION 1..214: note = Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 215 aa, synthetic construct
  REGION 1..215: note = Durvalumab light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC